(19) United States
(12) Patent Application Publication
(10) Pub. No.: US 2024/0051938 A1
(43) Pub. Date: Feb. 15, 2024

(54) SELECTIVE BRD4-DEGRADER MOLECULES

(71) Applicant: Regents of the University of Minnesota, Minneapolis, MN (US)

(72) Inventors: Huarui Cui, Minneapolis, MN (US); Anand Divakaran, Minneapolis, MN (US); Daniel A. Harki, Minneapolis, MN (US); William C. K. Pomerantz, Minneapolis, MN (US)

(73) Assignee: Regents of the University of Minnesota, Minneapolis, MN (US)

(21) Appl. No.: 18/228,422

(22) Filed: Jul. 31, 2023

Related U.S. Application Data
(60) Provisional application No. 63/394,115, filed on Aug. 1, 2022.

(51) Int. Cl. C07D 401/14; A61P 35/00

(52) U.S. Cl. CPC C07D 401/14; A61P 35/00

(57) ABSTRACT

The invention provides a compound of formula (I):

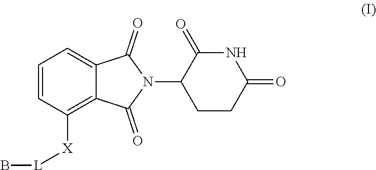

or a salt thereof wherein B, L, and X have any of the values defined in the specification, as well as compositions comprising a compound of formula (I) or a salt thereof. The compounds selectively degrade BRD4, and are useful as to treat cancer and inflammatory conditions.

### PRIORITY

This application claims priority to U.S. Provisional Application No. 63/394,115, filed 1 Aug. 2022. The entire contents of this United States Provisional patent application are hereby incorporated herein by reference.

### BACKGROUND

Bromodomain and Extra-Terminal (BET) family proteins, BRD2, 3, 4 and testis-specific protein, BRDT, are essential epigenetic regulators of gene expression through molecular recognition of acetylated protein substrates (Muller, S., et al., Expert Rev. Mol. Med. 2011, 13 (September), e29). Of the three ubiquitously expressed BET proteins, BRD4 is accepted as the most disease-relevant target as numerous preclinical and clinical investigations have identified its essential role in cancer and inflammatory diseases (Nabet, B, et al., Nat. Chem. Biol. 2018, 14 (5), 431-441; and Runcie, A. C., et al., Chem. Sci. 2018, 9 (9), 2452-2468), including through regulation of oncogenes like MYC (Delmore, J. E, et al., Cell 2011, 146 (6), 904-917; and Mertz, J. A, et al., Proc. Natl. Acad. Sci. 2011, 108 (40), 16669-16674). The tandem bromodomains (BD1 and BD2) are the most ligandable domains of BET proteins, but selectivity remains a significant challenge due to their high degree of homology (Filippakopoulos, P, et al., Nature 2010, 468 (7327), 1067-1073; and Filippakopoulos, P, et al., Cell 2012, 149 (1), 214-231). In binding all eight BET bromodomains, pan-BET inhibitors have shown promising therapeutic efficacy but are plagued by significant dose limiting toxicities. More recently, selective inhibitors targeting either BD1 or BD2 of BET proteins have been developed and are better tolerated (Faivre, E. J, et al., Nature 2020, 578 (7794), 306-310; and Gilan, 0, et al., Science, 2020, 368 (6489), 387-394). Alternatively, selective inhibitor development for single BET bromodomains remains challenging.

As an alternative to inhibition, targeted protein degradation has emerged as a therapeutic modality leveraging event-driven pharmacology over traditional target occupancy models (Lai, A. C, et al., Nature Publishing Group Feb. 2, 2017, pp 101-114). Targeted degraders co-opt cellular proteostasis machinery, inducing proximity between cellular E3-ligases and proteins of interest, resulting in proteasomal degradation of target proteins. Given their sub-stoichiometric mode of action, modularity in design and promise of targeting traditionally ‘undruggable’ targets, considerable effort has been devoted to the design of novel degrader molecules from a wide variety of protein classes including BET proteins (Feral, A, et al., Advances. Adv. Ther. 2020, 3 (11), 2000148; Vogelmann, A, et al., Current Opinion in Chemical Biology. Elsevier Ltd Aug. 1, 2020, pp 8-16; and Flanagan, J. J.; Neklesa, T. K. Targeting Nuclear Receptors with PROTAC Degraders. Molecular and Cellular Endocrinology. Elsevier Ireland Ltd Aug. 1, 2019, p 110452). Although BET-degraders have improved therapeutic efficacy compared to pan-BET inhibitors and elicit greater phenotypic responses than BET-inhibition alone (Yang, C. Y, et al., Small-Molecule PROTAC Degraders of the Bromodomain and Extra Terminal (BET) Proteins—A Review. Drug Discovery Today: Technologies. Elsevier Ltd Apr. 1, 2019, pp 43-51), both face challenges with dose-limiting toxicities including thrombocytopenia attributed to targeting BRD3 and BRD2 which play compensatory roles (Stonestrom, A. J, et al., Blood 2015, 125 (18), 2825-2834). Given the overlapping functions of BET-family proteins, pleiotropic effects observed with pan-BET degraders confound interpretation of the role individual BET-proteins play as drivers of disease.

While selective degraders have been developed from promiscuous ligands (Bondeson, D. P, et al., Cell Chem. Biol. 2018, 25 (1), 78-87.e5), this remains a non-trivial endeavor. To selectively degrade individual BET-family members, current degraders are based on pan-BET inhibitors and therefore lack high selectivity. MZ1 and dBET1 (FIG. 1A) degrade BRD4 with <10-fold selectivity over BRD2 and 3, primarily through favorable BRD4 dissociation kinetics (Zengerle, M.; Chan, K. H.; Ciulli, A. Selective Small Molecule Induced Degradation of the BET Bromodomain Protein BRD4. ACS Chem. Biol. 2015, 10 (8), 1770-1777; and Winter, G. E, et al., Science, 2015, 348 (6241), 1376-1381). By optimizing cooperativity, ternary complex formation and linker geometry of pan-BET ligands with BRD4, AT1, ZXH-3-26 and KB02-JQ1 (FIG. 1B) do show higher BRD4 selectivity, including by higher sensitivity chemoproteomic methods (Gadd, M. S., et al., Nat. Chem. Biol. 2017, 13 (5), 514-521; Nowak, R. P, et al., Nat. Chem. Biol. 2018, 14 (7), 706-714; and Zhang, X, et al., Nat. Chem. Biol. 2019, 15 (7), 737-746). However, these results indicate a snapshot in time that may not be representative over extended dosing periods or biological contexts and these designs are not generalizable with different linkers and E3-ligase ligands. An unaddressed concern in these cases is the reversible bromodomain inhibition of undegraded BET-proteins by the pan-BET ligand; although the degrader is optimized for ternary complex formation with BRD4, residual BET bromodomain binding has cellular effects in addition to BRD4 degradation and may result in undesired toxicity.

Currently there is a need for agents that are useful for selectively degrading BRD4 in cells.

### SUMMARY

A highly BRD4-BD1 selective inhibitor, iBRD4-BD1, with 23-6000-fold selectivity over ubiquitously expressed BET bromodomains has been identified. This inhibitor was used to test the efficiency of selective BRD4 degradation through BRD4-BD1 engagement alone. Design of dBRD4-BD1, demonstrated sustained BRD4 degradation, and led to a moderate increase in BRD2 and BRD3 protein levels. These results highlight the utility of domain-selective BET inhibitors and demonstrate BRD4-BD1 as a suitable target for BRD4 degradation.

The present invention provides proteolytic targeting chimeric molecules that induce selective degradation of the bromodomain and the extraterminal (BET) family of protein, BRD4. The compounds are useful for treating cancer and inflammatory conditions and for investigating the role of BRD4 in these and other conditions.

In one aspect the present invention provides compound of formula (I)

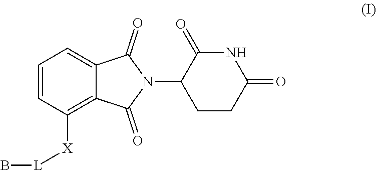

or a salt thereof wherein:

B is the residue of a molecule that selectively degrades BRD4 through its N-terminal bromodomain;

L is absent or a linker; and

X is O or N(R^{a}); and

IV is H or (C₁-C₆)alkyl.

The invention also provides a pharmaceutical composition comprising a compound of formula (I) or a pharmaceutically acceptable salt thereof, and a pharmaceutically acceptable excipient.

The invention also provides a method for treating cancer in an animal (e.g., a mammal such as a human) comprising administering a compound of formula (I) or a pharmaceutically acceptable salt thereof to the animal.

The invention also provides a method for treating an inflammatory condition in an animal (e.g., a mammal such as a human) comprising administering a compound of formula (I) or a pharmaceutically acceptable salt thereof to the animal.

The invention also provides a method for treating alcoholic hepatitis in an animal (e.g., a mammal such as a human) comprising administering a compound of formula (I) or a pharmaceutically acceptable salt thereof to the animal.

The invention also provides a compound of formula (I) or a pharmaceutically acceptable salt thereof for use in medical therapy.

The invention also provides a compound of formula I or a pharmaceutically acceptable salt thereof for the prophylactic or therapeutic treatment of cancer.

The invention also provides a compound of formula I or a pharmaceutically acceptable salt thereof for the prophylactic or therapeutic treatment of an inflammatory condition.

The invention also provides a compound of formula I or a pharmaceutically acceptable salt thereof for the prophylactic or therapeutic treatment of alcoholic hepatitis.

The invention also provides the use of a compound of formula I or a pharmaceutically acceptable salt thereof to prepare a medicament for treating cancer in an animal (e.g. a mammal such as a human).

The invention also provides the use of a compound of formula I or a pharmaceutically acceptable salt thereof to prepare a medicament for treating an inflammatory condition in an animal (e.g. a mammal such as a human).

The invention also provides the use of a compound of formula I or a pharmaceutically acceptable salt thereof to prepare a medicament for treating alcoholic hepatitis in an animal (e.g. a mammal such as a human).

The invention also provides processes and intermediates disclosed herein that are useful for preparing a compound of formula I or a salt thereof.

### BRIEF DESCRIPTION OF THE DRAWINGS

FIG. 1A-1C: Approaches to design selective BRD4 degraders. (A) pan-BET degraders with preference for BRD4 degradation. (B) Reported degraders with selectivity for degrading BRD4 over BRD2/BRD3 by optimizing pan-BET ligands for BRD4 binding kinetics and linkers. (C) This work focuses on selective BRD4 degradation through engaging its N-terminal bromodomain over other BET bromodomains.

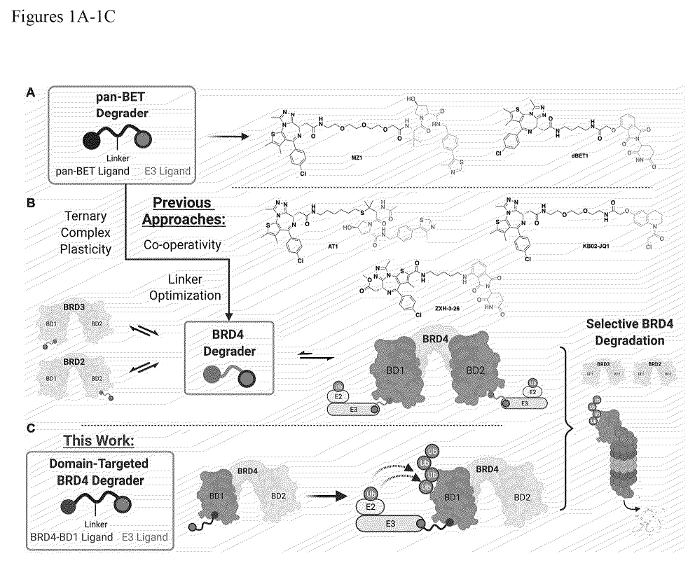

FIG. 2A-2E: Role of BD1 in BRD4 degradation and biophysical characterization of iBRD4-BD1. (A) Competition of pan-BET degrader, MZ1, with pan-BD1 or pan-BD2 inhibitors, iBET-BD1/BD2. (B) Trisubstituted imidazoles with BET-bromodomain selectivity for BRD4-BD1. (C) Structure of iBRD4-BD1, with variation in substitution pattern on northern aryl ring (D) Commercial Alphascreen assay with BET-bromodomains from Reaction Biology.

Data reported as mean of duplicate experiments. (E) Isothermal dose-response CETSA demonstrating target engagement of BRD4 in MM.1S cells after 1 hour treatment.

FIG. 3A-3B: Biophysical characterization of dBRD4-BD1. (A) Structure of dBRD4-BD1. (B) Alphascreen binding assay with 9xHis-BRD4-BD1, reported as mean±SD of three independent trials performed in duplicate.

FIG. 4A-4D: Degradation of BRD4 in MM.1S cells. (A) Representative blots for degradation of BRD4 after 24-hour treatment. (B) Quantified densitometry for three biological replicates of blots from (A). (C) Time-course study of BRD4 degradation by dBRD4-BD1. (D) Rescue of BRD4 degradation using pan-BD1, proteasome and neddylation inhibitors, but not pan-BD2 inhibitor, iBET-BD2.

FIG. 5: 72 hour Alamar blue cytotoxicity assay of MM.1S cells with compounds. Data reported as mean±SD of three independent trials of three replicates each.

FIG. 6: Shows full images of western blots.

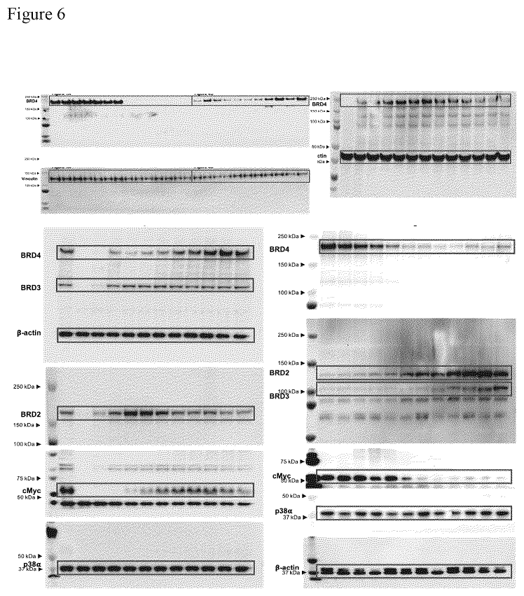

FIG. 7: Shows florescence-anisotropy data for Compounds 100 and 120.

FIG. 8: Shows Western-blot data for Compounds 100 and 120.

### DETAILED DESCRIPTION OF THE INVENTION

The following definitions are used, unless otherwise described: Alkyl denotes both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g. tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc.), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc.). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “alkoxycarbonyl” as used herein refers to a group (alkyl)—O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P.G.M. Wuts and T.W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

In one embodiment X is O or NH.

### Linker

In one embodiment the linker is absent. The linker can vary in length and atom composition and for example can be branched or non-branched or cyclic or a combination thereof. The linker may also modulate the properties of the compound of formula (I) such as but not limited to solubility, stability, and aggregation.

In one embodiment the linker comprises about 3-500 atoms. In one embodiment the linker comprises about 3-400 atoms. In one embodiment the linker comprises about 3-200 atoms. In one embodiment the linker comprises about 3-100 atoms. In one embodiment the linker comprises about 3-75 atoms. In one embodiment the linker comprises about 3-50 atoms. In one embodiment the linker comprises about 3-25 atoms. In one embodiment the linker comprises about 3-15 atoms. In one embodiment the linker comprises about 3-10 atoms. In one embodiment the linker comprises about 3-5 atoms.

In one embodiment the linker comprises atoms selected from H, C, N, S and O.

In one embodiment the linker comprises atoms selected from H, C, N, and 0.

In one embodiment the linker comprises a branched or unbranched, saturated or unsaturated, hydrocarbon chain, having from about 1 to 100 (or 1-75, 1-50, 1-25, 1-10, 1-5, 1-3, 5-75, 5-50, 5-25, 5-10, or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R^{a} is independently H or (C₁-C₆)alkyl.

In one embodiment the linker comprises a branched or unbranched, saturated or unsaturated, hydrocarbon chain, having from about 1 to 100 (or 1-75, 1-50, 1-25, 1-10, 1-5, 1-3, 5-75, 5-50, 5-25, 5-10, or 2-5 carbon atoms) wherein one or more of the carbon atoms is optionally substituted with oxo (═O); and wherein one or more of the carbon atoms is optionally replaced independently by —O—, —S, or —N(R^{a})—, wherein each R^{a} is independently H or (C₁-C₆)alkyl.

In one embodiment the linker comprises a branched or unbranched, saturated or unsaturated, hydrocarbon chain, having from about 1 to 100 (or 1-75, 1-50, 1-25, 1-10, 1-5, 1-3, 5-75, 5-50, 5-25, 5-10, or 2-5 carbon atoms) wherein one or more of the carbon atoms is optionally substituted with oxo (═O); and wherein one or more of the carbon atoms is optionally replaced independently by —O— or —N(R^{a})—, wherein each R^{a} is independently H or (C₁-C₆)alkyl.

In one embodiment the linker comprises a branched or unbranched, saturated or unsaturated, hydrocarbon chain, having from about 2 to 10 carbon atoms wherein one or more of the carbon atoms is optionally substituted with oxo (═O); and wherein one or more of the carbon atoms is optionally replaced independently by —O— or —N(R^{a})—, wherein each R^{a} is independently H or (C₁-C₆)alkyl.

In one embodiment the linker comprises a branched or unbranched, saturated or unsaturated, hydrocarbon chain, having from about 2 to 10 carbon atoms wherein one or more of the carbon atoms is optionally substituted with oxo (═O); and wherein one or more of the carbon atoms is optionally replaced independently by —O— or —N(R^{a})—, wherein each R^{a} is independently H or (C₁-C₆)alkyl.

In one embodiment the linker comprises a polyethylene glycol. In one embodiment the linker comprises a polyethylene glycol linked to the remainder of the targeted conjugate by a carbonyl group. In one embodiment the polyethylene glycol comprises about 1 to about 50 or about 5 to about 50 or about 3 to about 10 repeat (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

In one embodiment the linker is selected from the group consisting of —(CH₂)ₙ—C(═O)—, and —(CH₂CH₂O)ₚ—(CH₂)ᵣ—NH—C(═O)—; wherein n is 2, 3, 4, 5, or 6; p is 2, 3, 4, 5, or 6; and r is 2, 3, 4, 5, or 6.

In one embodiment the linker is selected from the group consisting of —(CH₂)₅—C(═O)—, and —(CH₂CH₂O)₂—(CH₂)₂—NH—C(═O)—.

Molecules that selectively degrade BRD4 through its N-terminal bromodomain

In one embodiment, B is the residue of a molecule that binds to BRD4 with at least 10-fold selectivity against the other BET proteins.

In one embodiment, B is the residue of a molecule that binds to BRD4 with at least 100-fold selectivity against the other BET proteins.

In one embodiment, B is the residue of a molecule that binds to BRD4 with at least 1000-fold selectivity against the other BET proteins.

In one embodiment, B is a residue of:

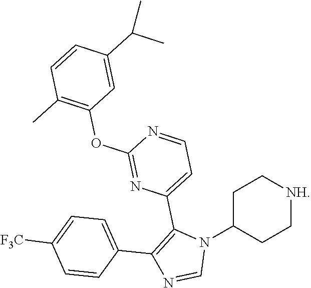 [SMILES: Cc1ccc(C(C)C)cc1Oc1nccc(-c2c(-c3ccc(C(F)(F)F)cc3)ncn2C2CCNCC2)n1]

In one embodiment, B is a residue of:

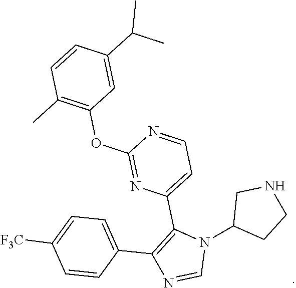 [SMILES: Cc1ccc(C(C)C)cc1Oc1nccc(-c2c(-c3ccc(C(F)(F)F)cc3)ncn2C2CCNC2)n1]

Such a compound can be prepared using a procedure similar to that described in Example 1 for the preparation of Compound 6.

In one embodiment, the invention provides the compound:

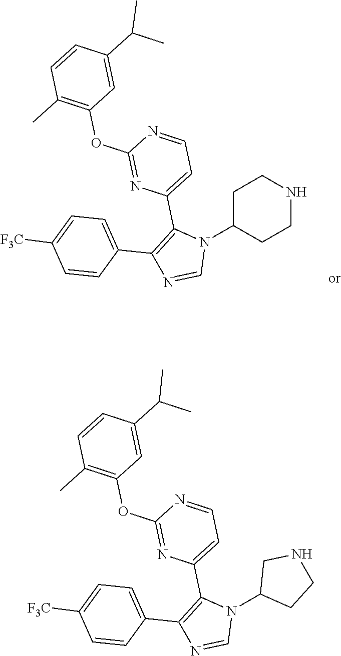 [SMILES: Cc1ccc(C(C)C)cc1Oc1nccc(-c2c(-c3ccc(C(F)(F)F)cc3)ncn2C2CCNCC2)n1 ; Cc1ccc(C(C)C)cc1Oc1nccc(-c2c(-c3ccc(C(F)(F)F)cc3)ncn2C2CCNC2)n1]

or a salt thereof. Such compounds and salts have useful BRD4 degrading properties and they are useful intermediates for preparing compounds of formula (I).

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine) followed by the removal of 1 or more atoms from the second functional group to create the open valence.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### EXAMPLES

All commercially available chemicals were used without further purification. Flash column chromatography was performed on a Teledyne-Isco Rf-plus CombiFlash instrument with RediSep Gold Silica columns. Spectra were collected on a Bruker Avance III HD 500 or a Bruker Avance III HD 900. Chemical shifts (6) are reported in parts per million (ppm) and referenced to residual solvent signal, ¹H 7.26 ppm, ¹³C 77.0 ppm in CDCl₃; 41 3.32 ppm, ¹³C 49.2 ppm in MeOD; ¹H 2.50 ppm, ¹³C 39.5 ppm in DMSO-d6. Coupling constants (J) are reported in Hz. Splitting patterns are reported as s (singlet), d (doublet), t (triplet), q (quartet), m (multiplet) and br (broad). High resolution mass spectrometry was used with positive-mode electrospray-ionization methods (ESI-MS) using a Bruker BioTOF II. Purities of final compounds were verified by reverse-phase high-performance liquid chromatography (RP-HPLC) with a Zorbax C-18 column across a 10-60% gradient of 0.1% TFA water:acetonitrile over 60 minutes.

### Example 1. Preparation of Compound 8 (dBRD4-BD1)

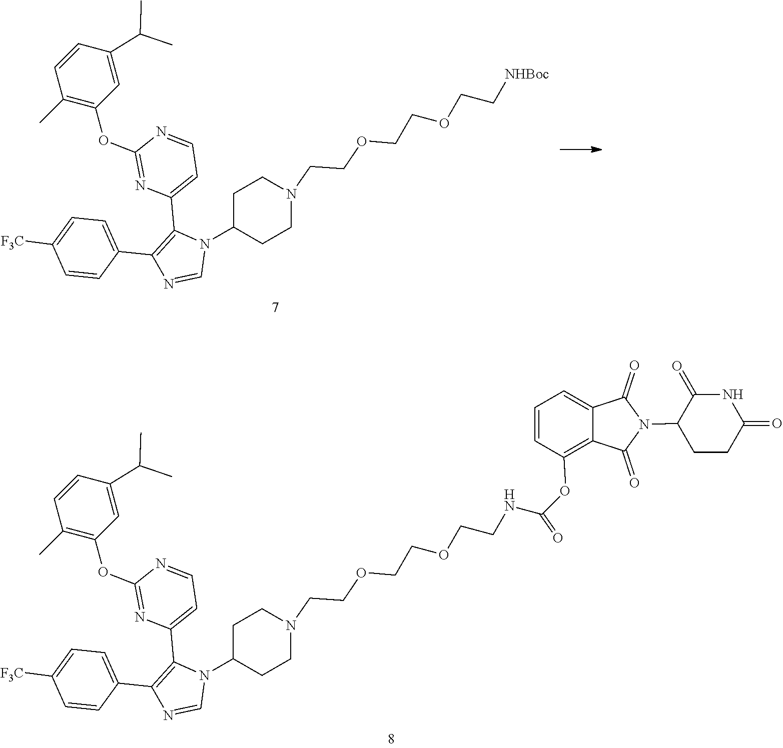 [SMILES: Cc1ccc(C(C)C)cc1Oc1nccc(-c2c(-c3ccc(C(F)(F)F)cc3)ncn2C2CCN(CCOCCOCCNC(=O)OC(C)(C)C)CC2)n1 ; Cc1ccc(C(C)C)cc1Oc1nccc(-c2c(-c3ccc(C(F)(F)F)cc3)ncn2C2CCN(CCOCCOCCNC(=O)Oc3cccc4c3C(=O)N(C3CCC(=O)NC3=O)C4=O)CC2)n1]

Compound 7 (11 mg, 15 μM) was dissolved in 2 M HCl in dioxane (2 mL) and stirred for 16 hours at room temperature. The reaction mixture was dried under a stream of nitrogen and dried further under vacuum. The crude material was dissolved in dry DMF (900 μL) and DIEA (7.5 mg, 60 μM) was added while stirring. A mixture of acid 3 (10 mg, 30 μM) and HCTU (12 mg, 30 μM) was subsequently added in dry DMF (100 μL) and the reaction mixture was stirred for 2 hours at room temperature. The crude material was purified by preparative HPLC (10-75% gradient in 0.1% aqueous TFA:acetonitrile) to yield the desired product. 1H NMR (900 MHz, DMSO-d6) δ 11.1 (s, 1H), 8.7 (d, J=5.0 Hz, 1H), 8.1 (s, 1H), 8.0 (t, J=5.7 Hz, 1H), 7.8 (t, J=8.1, 7.6 Hz, 1H), 7.7 (d, J=8.0 Hz, 2H), 7.6 (d, J=8.0 Hz, 2H), 7.5 (d, J=7.2 Hz, 1H), 7.4 (d, J=8.6 Hz, 1H), 7.2 (d, J=7.9 Hz, 1H), 7.2 (d, J=5.0 Hz, 1H), 7.1 (d, J=8.8 Hz, 2H), 5.1 (dd, J=13.0, 5.5 Hz, 1H), 4.8 (s, 2H), 4.4 (dqd, J=12.0, 7.8, 4.7, 4.1 Hz, 1H), 3.8 (t, J=4.9 Hz, 3H), 3.6 (dd, J=6.0, 3.4 Hz, 4H), 3.6 (dd, J=6.0, 3.2 Hz, 4H), 3.5 (t, J=5.7 Hz, 4H), 3.3 (q, J=5.7 Hz, 3H), 3.3-3.3 (m, 2H), 3.2 (s, 1H), 3.1-3.1 (m, 1H), 2.9-2.9 (m, 3H), 2.8 (h, J=6.9 Hz, 1H), 2.6 (dt, J=17.0, 3.5 Hz, 1H), 2.2-2.2 (m, 2H), 2.1-2.0 (m, 1H), 1.9-1.9 (m, 1H), 1.2 (s, 3H), 1.1 (d, J=7.0 Hz, 6H). Note: ¹H-NMR contained an acetone impurity @ 2.1 ppm. ¹³C NMR (226 MHz, DMSO-d6) δ 173.3, 170.4, 167.4, 167.2, 166.0, 165.2, 161.7, 159.6, 158.3, 155.4, 151.4, 148.3, 140.2, 138.4, 137.4, 133.5, 131.5, 128.4, 127.4, 125.9, 123.8, 120.9, 120.5, 118.0, 117.3, 116.6, 70.1, 69.8, 69.2, 68.0, 64.8, 55.8, 51.8, 51.0, 49.3, 40.7, 40.4, 39.3, 39.2, 39.1, 38.8, 33.3, 31.4, 31.2, 30.3, 29.5, 24.2, 22.5, 16.1. ¹⁹F NMR (471 MHz, Chloroform-d) δ−62.8, −75.8 (3xTFA salt). HRMS (ESI-TOF) m/z [M+H]⁺ calcd. for C₅₀H₅₄F₃N₈O₉ 967.3965, found 967.3900.

The intermediate acid 3 was prepared as follows.

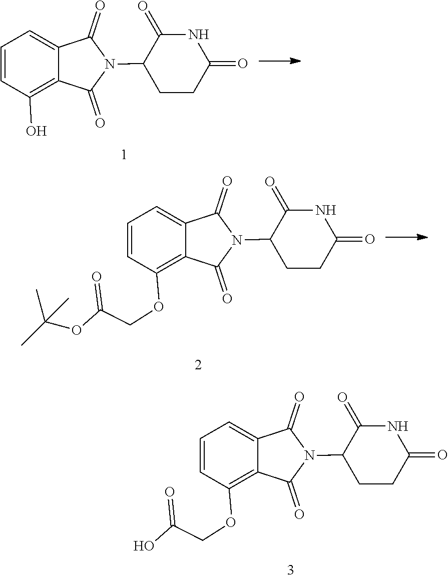 [SMILES: O=C1CCC(N2C(=O)c3cccc(O)c3C2=O)C(=O)N1 ; CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

a. Preparation of Compound 2. In a 10 mL round-bottomed flask, compound 1 (120 mg, 0.43 mM) was added to dry DMF (1 mL), followed by addition of potassium carbonate (Alfa Aesar, 90 mg, 0.65 mM) and tert-butyl 2-bromoacetate (Sigma, 93 mg, 0.48 mM). The reaction mixture was stirred at room temperature and followed by TLC. After two hours, the reaction was quenched by addition of water (10 mL) and the yellow aqueous layer was extracted 3×in ethyl acetate (10 mL). Longer reaction times can lead to a dialkylated product. The combined organics were dried over brine and anhydrous magnesium sulfate, concentrated by rotary evaporation, and purified by Combiflash chromatography (0-100% hexane:ethyl acetate) to yield a yellow solid (86 mg, 52%). ¹H NMR (500 MHz, Methanol-d4) δ 7.8 (dd, J=8.5, 7.3 Hz, 1H), 7.5 (dd, J=7.3, 0.6 Hz, 1H), 7.3 (d, J=8.4 Hz, 1H), 5.1 (dd, J=12.5, 5.5 Hz, 1H), 4.9 (s, 2H), 2.9-2.8 (m, 2H), 2.8-2.7 (m, 2H), 2.2-2.1 (m, 1H), 1.5 (s, 9H).
b. Preparation of Compound 3. Compound 2 (80 mg, 0.2 mM) was dissolved in dry DCM (2 mL) and trifluoracetic acid (2 mL) was added dropwise while stirring. After stirring at room temperature for 2 hours and confirming completion of reaction progress by TLC, the reaction mix was blown dry under a stream of nitrogen and dried under vacuum. The crude product, 3, was used without further purification.

The intermediate Compound 7 was prepared as follows.

c. Preparation of Compound 5.

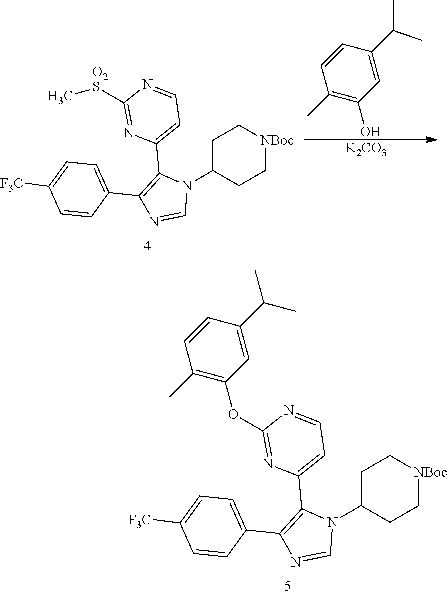

Compound 4 was synthesized as previously described (Cui, H., et al., Angew. Chemie-Int. Ed. 2021, 60 (3), 1220-1226). In a sealed tube, Compound 4 (310 mg, 0.56 mM) was stirred with carvacrol (Sigma, 250 mg, 1.7 mM), potassium carbonate (150 mg, 1.1 mM) and 2 mL DMF for 16 hours at 130° C. behind a blast shield. The reaction mixture was cooled before being quenched in 20 mL cold water and extracted 3× in 40 mL ethyl acetate. The combined organics were dried over brine and anhydrous magnesium sulfate and concentrated by rotary evaporation. The crude brown oil was purified by normal phase Combiflash chromatography (0-100% hexane:ethyl acetate) to yield a yellow foam (340 mg, 96% yield). ¹H NMR (400 MHz, Chloroform-d) δ 8.4 (d, J=5.1 Hz, 1H), 7.8 (s, 1H), 7.7-7.5 (m, 4H), 7.2 (d, J=7.8 Hz, 2H), 7.1 (d, J=7.8, 1.8 Hz, 1H), 7.1 (d, J=1.8 Hz, 1H), 6.9 (d, J=5.1 Hz, 1H), 4.7-4.6 (m, 1H), 4.1 (q, J=7.1 Hz, 2H), 3.0-2.9 (m, 1H), 2.5 (t, J=12.8 Hz, 2H), 2.2 (s, 3H), 2.1 (s, 1H), 2.0-1.9 (m, 2H), 1.8-1.7 (m, 3H), 1.5 (s, 9H), 1.3 (d, J=6.9 Hz, 7H). ¹³C NMR (101 MHz, Chloroform-d) δ 165.1, 160.2, 159.8, 154.4, 151.2, 148.4, 137.9, 136.8, 131.2, 128.8, 127.5, 125.6, 125.6, 125.5, 124.0, 120.2, 116.3, 80.1, 54.1, 33.6, 28.4, 23.9, 16.2. ¹⁹F NMR (471 MHz, Chloroform-d) δ−64.1.

d. Preparation of Compound 6.

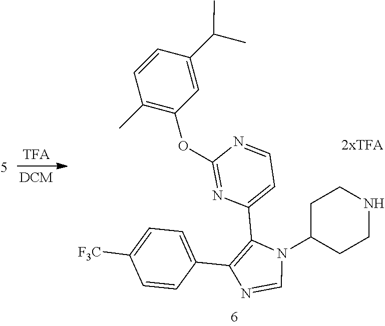 [SMILES: Cc1ccc(C(C)C)cc1Oc1nccc(-c2c(-c3ccc(C(F)(F)F)cc3)ncn2C2CCNCC2)n1]

Compound 5 (310 mg, 0.50 mM) was dissolved in DCM (5 mL) and TFA (1 mL) was added dropwise while stirring. The reaction mix was stirred overnight at room temperature and blown dry under a stream of nitrogen before being dried under vacuum. The crude material was triturated in cold diethyl ether to yield a fine white powder that was used without further purification (313 mg, 83% yield). ¹H NMR (500 MHz, Methanol-d4) δ 8.5 (d, J=5.0, 1.5 Hz, 1H), 8.2 (s, 1H), 7.7 (d, J=8.1 Hz, 2H), 7.6 (d, J=8.0 Hz, 2H), 7.3 (d, J=7.7 Hz, 1H), 7.1 (d, J=7.8 Hz, 2H), 7.0 (d, J=5.2 Hz, 1H), 4.7 (dt, J=12.1, 4.0 Hz, 1H), 3.5 (q, J=7.1 Hz, 2H), 2.9 (p, J=6.9 Hz, 1H), 2.8 (dt, J=12.4 Hz, 2H), 2.2 (s, 3H), 2.1 (qd, J=13.1, 4.0 Hz, 2H), 1.3 (d, J=7.1, 1.5 Hz, 6H), 1.2 (t, J=7.0, 1.5 Hz, 3H). ¹³C NMR (125 MHz, Methanol-d4). ¹³C NMR (126 MHz, Methanol-d4) δ 164.9, 160.6, 158.6, 151.2, 148.6, 137.0, 136.4, 131.1, 129.0, 127.2, 125.5, 123.6, 120.0, 116.8, 51.7, 43.3, 33.4, 29.6, 23.0, 14.8. ¹⁹F NMR (471 MHz, Methanol-d4) δ-64.2, −75.8 (2xTFA salt). HRMS (ESI-TOF) m/z [M+H]⁺ calcd. for C₂₉H₃₁F₃N₅₀ 522.2480, found 522.2443.

e. Preparation of Compound 7.

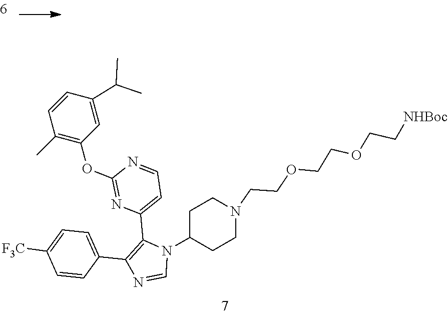 [SMILES: Cc1ccc(C(C)C)cc1Oc1nccc(-c2c(-c3ccc(C(F)(F)F)cc3)ncn2C2CCN(CCOCCOCCNC(=O)OC(C)(C)C)CC2)n1]

In a sealed tube, compound 6 (55 mg, 0.1 mM) was stirred with tert-butyl 3-(2-(2-bromoethoxy)ethoxy)propanoate (PurePEG, 49 mg, 0.2 mM), DIEA (27 mg, 0.2 mM) and 1 mL 1,4-dioxane for 16 hours at 120° C. behind a blast shield. The reaction mixture was cooled before being quenched in 10 mL of cold water and extracted 3× in 10 mL ethyl acetate. The combined organics were dried over anhydrous magnesium sulfate and concentrated by rotary evaporation. The crude orange oil was purified by Combiflash chromatography (0-25% DCM:methanol) to yield an off-white powder (13 mg, 17% yield). ¹H NMR (500 MHz, DMSO-d6) δ 8.6 (d, J=5.0 Hz, 1H), 8.2 (s, 1H), 7.7 (d, J=8.2 Hz, 2H), 7.6 (d, J=8.1 Hz, 2H), 7.2 (d, J=8.3 Hz, 1H), 7.1 (d, J=5.1 Hz, 1H), 7.1 (dd, J=5.8, 2.0 Hz, 2H), 6.7 (t, J=5.8 Hz, 1H), 4.2-4.1 (m, 2H), 3.5 (s, 6H), 3.4 (t, J=6.2 Hz, 4H), 3.2 (d, J=3.4 Hz, 3H), 3.1 (q, J=6.0 Hz, 2H), 2.9-2.8 (m, 3H), 2.1 (s, 3H), 1.9-1.8 (m, 4H), 1.7 (dd, J=9.0, 4.7 Hz, 2H), 1.4 (s, 9H), 1.2 (d, J=6.9 Hz, 6H). Note: ¹H-NMR contained a minor dioxane impurity @ 3.5 ppm. ¹³C NMR (126 MHz, DMSO-d6) δ 165.2, 161.6, 159.9, 151.4, 148.3, 140.4, 138.7, 138.2, 131.6, 128.5, 127.3, 125.8, 125.5, 123.8, 120.5, 117.8, 70.1, 69.9, 69.6, 69.0, 57.3, 54.0, 53.1, 33.3, 33.0, 29.5, 28.7, 24.2, 16.2. ¹⁹F NMR (471 MHz, DMSO-d6) δ−60.9. HRMS (ESI-TOF) m/z [M+H]⁺ calcd. for C₄₀H₅₂F₃N₆O₅ 753.3951, found 753.4131.

### Example 2. Preparation of Compound 100

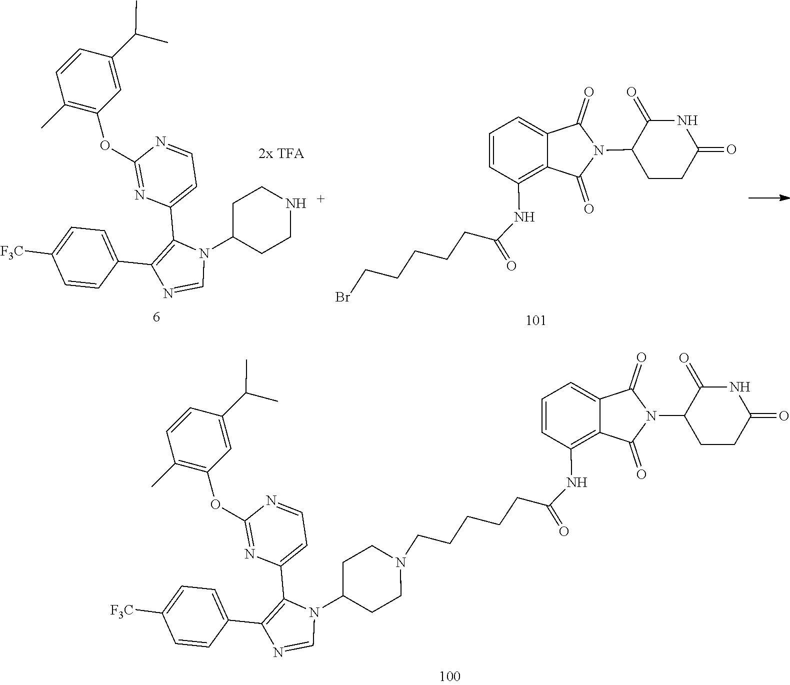 [SMILES: Cc1ccc(C(C)C)cc1Oc1nccc(-c2c(-c3ccc(C(F)(F)F)cc3)ncn2C2CCNCC2)n1 ; O=C1CCC(N2C(=O)c3cccc(NC(=O)CCCCCBr)c3C2=O)C(=O)N1 ; Cc1ccc(C(C)C)cc1Oc1nccc(-c2c(-c3ccc(C(F)(F)F)cc3)ncn2C2CCN(CCCCCC(=O)Nc3cccc4c3C(=O)N(C3CCC(=O)NC3=O)C4=O)CC2)n1]

1,4-Dioxane (2.5 mL), salt 6 (17 mg, 20 and DIEA (8 mg, 60 μM) were added to an evacuated round-bottomed flask equipped with a nitrogen-filled balloon. The mixture was stirred at room temperature for 5 minutes, followed by addition of compound 101 (11 mg, 25 μM). The reaction mixture was stirred at 60° C. for 16 hours, cooled and concentrated by rotary evaporation. The crude product was purified by preparative HPLC (10-75% gradient in 0.1% aqueous TFA:acetonitrile) to yield the desired product. ¹H NMR (500 MHz, Methanol-d4) δ 8.7 (d, J=8.4 Hz, 1H), 8.5 (d, J=5.1 Hz, 1H), 8.2 (s, 1H), 7.8 (dd, J=8.4, 7.4 Hz, 1H), 7.7 (d, J=8.1 Hz, 2H), 7.6 (d, J=8.0 Hz, 3H), 7.3 (dd, J=7.9, 5.2 Hz, 1H), 7.2-7.1 (m, 2H), 7.0 (d, J=5.1 Hz, 1H), 5.2 (dd, J=12.5, 5.5 Hz, 1H), 4.8-4.7 (m, 1H), 3.6 (d, J=12.6 Hz, 2H), 3.2 (d, J=8.4 Hz, 2H), 3.0-2.8 (m, 2H), 2.8 (tdd, J=21.0, 8.6, 5.6 Hz, 4H), 2.6 (t, J=7.2 Hz, 2H), 2.3 (d, J=13.5 Hz, 2H), 2.2 (s, 3H), 2.2-2.2 (m, 1H), 1.9-1.8 (m, 4H), 1.6 (p, J=7.7 Hz, 2H), 1.3 (d, J=6.9 Hz, 7H). ¹³C NMR (126 MHz, MeOD) δ 173.04, 172.74, 169.92, 168.55, 166.78, 164.95, 160.51, 158.91, 151.23, 148.55, 141.49, 136.86, 136.73, 135.75, 131.71, 131.06, 129.14, 128.94, 127.31, 125.55, 125.44, 125.25, 123.67, 119.97, 118.21, 116.83, 56.64, 51.68, 51.46, 49.19, 36.25, 33.41, 30.73, 30.22, 25.54, 24.14, 23.64, 22.98, 22.23, 14.79. ¹⁹F NMR (471 MHz, MeOD) δ−64.18, −77.25. HRMS (ESI-TOF) m/z [M+H]⁺ calcd. for C₄₈H₅₀F₃N₈O₆ 891.3805, found 891.4654.

The intermediate compound 101 was prepared as follows.

a. Preparation of Compound 101.

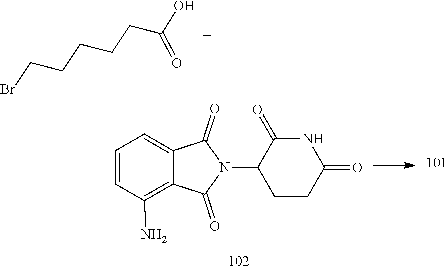 [SMILES: O=C(O)CCCCCBr ; Nc1cccc2c1C(=O)N(C1CCC(=O)NC1=O)C2=O]

To an evacuated round-bottomed flask equipped with a nitrogen-filled balloon, dry DCM (5 mL) and bromohexanoic acid (1.0 g, 5.1 mM) were added. This was followed by dropwise addition of thionyl chloride (0.73 g, 6.2 mM) and a single drop of dry DMF. The reaction mixture was stirred at room temperature for 2 hours, when evolution of bubbles ceased, and reaction progress was confirmed by TLC. The reaction mix was concentrated by rotary evaporation to leave a clear yellow oil. The crude material was diluted in dry THF (5 mL) and pomalidomide (102, 0.50 g, 2 mM) was added while stirring. The reaction mix was stirred overnight under refluxing conditions. After cooling and concentration by rotary evaporation, the product was purified by CombiFlash chromatography (0-15% DCM:methanol) to yield a white/pale yellow powder (720 mg, 79%). ¹H NMR (500 MHz, DMSO-d6) δ 11.2 (s, 1H), 9.7 (s, 1H), 8.5 (d, J=8.4 Hz, 1H), 7.8 (t, J=8.4, 7.3 Hz, 1H), 7.6 (d, J=7.3, 0.8 Hz, 1H), 5.2 (dd, J=12.9, 5.4 Hz, 1H), 3.5 (t, J=6.7 Hz, 2H), 3.0-2.8 (m, 1H), 2.7-2.5 (m, 2H), 2.1 (dtd, J=12.9, 5.3, 2.3 Hz, 1H), 1.8 (dt, J=14.9, 6.9 Hz, 2H), 1.7 (p, J=7.5 Hz, 2H), 1.5-1.4 (m, 2H).

### Example 3. Preparation of Compound 120

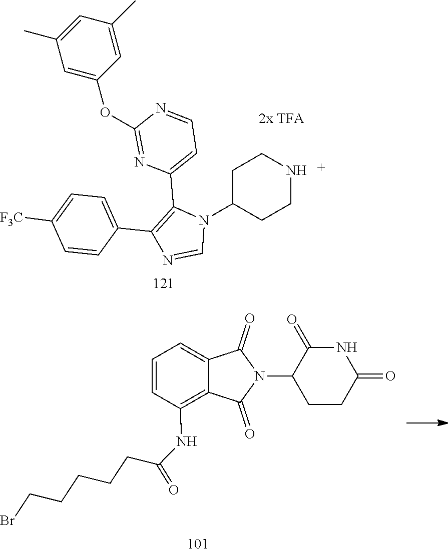 [SMILES: Cc1cc(C)cc(Oc2nccc(-c3c(-c4ccc(C(F)(F)F)cc4)ncn3C3CCNCC3)n2)c1 ; O=C1CCC(N2C(=O)c3cccc(NC(=O)CCCCCBr)c3C2=O)C(=O)N1]

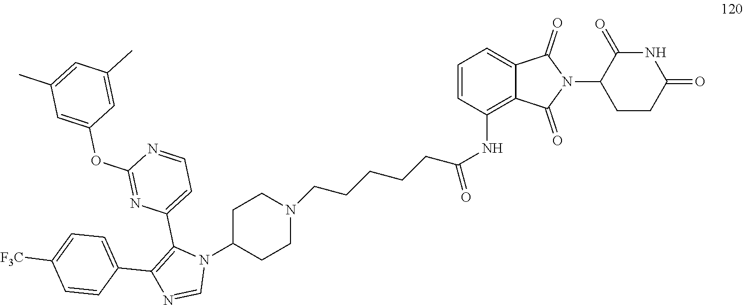 [SMILES: Cc1cc(C)cc(Oc2nccc(-c3c(-c4ccc(C(F)(F)F)cc4)ncn3C3CCN(CCCCCC(=O)Nc4cccc5c4C(=O)N(C4CCC(=O)NC4=O)C5=O)CC3)n2)c1]

A 50:50 mixture of dry DMF:acetonitrile (2 mL), salt 121 (25 mg, 50 μM), and anhydrous potassium carbonate (35 mg, 250 μM) were added to a dram vial and stirred at room temperature for 30 minutes. In a separate vial, sodium iodide (10 mg, 65 μM) and compound 101 (30 mg, 65 μM) were stirred at room temperature. The two mixtures were combined and stirred at room temperature for 16 hours. The reaction mix was filtered and concentrated by rotary evaporation, then purified by CombiFlash chromatography (0-20% DCM:methanol). The desired fractions were further purified by preparative HPLC (25-75% gradient in 0.1% aqueous TFA:acetonitrile) to yield the desired product. ¹H NMR (500 MHz, Methanol-d4) δ 9.2 (s, 1H), 8.7 (d, J=8.4 Hz, 1H), 7.9 (dd, J=8.4, 7.4 Hz, 1H), 7.9-7.8 (m, 1H), 7.8 (d, J=10.0 Hz, 1H), 7.7-7.6 (m, 2H), 7.6 (s, 1H), 7.2 (d, J=8.4 Hz, 1H), 7.1-7.1 (m, 1H), 7.0 (s, 1H), 6.9 (s, 2H), (dd, J=12.5, 5.5 Hz, 1H), 4.6 (tt, J=11.1, 4.9 Hz, 1H), 3.6 (t, J=6.2 Hz, 2H), 3.2-3.1 (m, 2H), 2.9 (ddd, J=18.0, 14.2, 5.3 Hz, 1H), 2.8-2.7 (m, 3H), 2.7 (s, 1H), 2.6 (t, J=7.2 Hz, 2H), 2.3 (s, 6H), 2.3 (s, 2H), 2.2-2.1 (m, 1H), 1.9 (h, J=5.9, 4.6 Hz, 4H), 1.5 (p, J=7.8 Hz, 2H), 1.3 (d, J=25.6 Hz, 1H). ¹³C NMR (126 MHz, MeOD) δ 173.09, 172.74, 169.92, 168.52, 164.09, 153.56, 143.19, 141.09, 139.59, 136.73, 135.73, 134.66, 131.71, 129.01, 126.61, 125.58, 121.25, 119.05, 118.19, 116.97, 112.86, 56.59, 52.84, 51.18, 49.19, 36.27, 30.74, 29.78, 25.59, 24.19, 23.54, 22.24, 20.02. ¹⁹F NMR (471 MHz, MeOD) δ−64.07, −76.94. HRMS (ESI-TOF) m/z [M+H]⁺ calcd. for C₄₇H₄₇F₃N₇O₆ 862.3539, found 862.4059.

The intermediate compound 121 was prepared as described by Cui, H., et al., Angew. Chemie-Int. Ed. 2020, 59, 2-9.

### Example 4

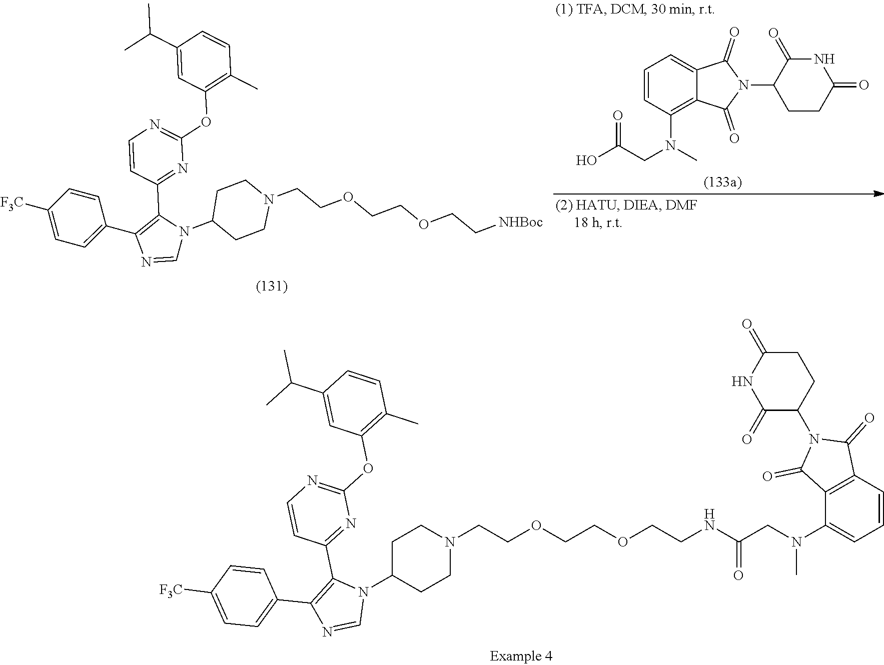 [SMILES: Cc1ccc(C(C)C)cc1Oc1nccc(-c2c(-c3ccc(C(F)(F)F)cc3)ncn2C2CCN(CCOCCOCCNC(=O)OC(C)(C)C)CC2)n1 ; CN(CC(=O)O)c1cccc2c1C(=O)N(C1CCC(=O)NC1=O)C2=O ; Cc1ccc(C(C)C)cc1Oc1nccc(-c2c(-c3ccc(C(F)(F)F)cc3)ncn2C2CCN(CCOCCOCCNC(=O)CN(C)c3cccc4c3C(=O)N(C3CCC(=O)NC3=O)C4=O)CC2)n1]

Intermediate (131) (41 mg, 0.053 mmol) was dissolved in a mixture of DCM (0.25 mL) and TFA (0.25 mL) and stirred for 30 minutes at ambient temperature. This mixture was then evaporated of volatiles under a stream of nitrogen before being dissolved a mixture of DMF (0.5 mL) containing excess DIEA (0.1 mL). Separately, compound (133a) (37 mg, 0.108 mmol) was dissolved in DMF (0.5 mL) and added to it was HATU (42 mg, 0.108 mmol) and DIEA (42 mg, mmol). This mixture containing activated (3a) was then added dropwise to the solution containing (131), and the combined mixture was allowed to stir for 18 hours at ambient temperature. The reaction mixture was then diluted with EtOAc before being washed with 10% LiCl_{(aq)}. The organic layer was isolated and dried under magnesium sulfate before being purified through column chromatography with a Teledyne CombiFlash instrument (0 to 30% MeOH in DCM, 4 g silica, 20 min). This resulted in a vibrant yellow solid (Example 4) (39.3 mg, 74%). 1 E1 NMR (400 MHz, DMSO) δ 11.09 (s, 1H), 8.65 (d, 1H, J=5.04 Hz), 8.31 (br, 1H), 8.13 (br, 1H), 7.66 (d, 2H, J=8.27 Hz), 7.59 (d, 2H, J=8.05 Hz), 7.22 (m, 3H), 7.12 (s, 1H), 7.04 (m, 2H)p, 5.07 (dd, 1H, J=12.85 and 5.40 Hz), 4.39 (q, 2H, J=5.31 Hz), 4.18 (d, 2H, J=3.76 Hz), 3.49 (br, 4H), 3.41 (t, 2H, J=7.32 Hz), 3.20 (q, 2H, J=5.91 ppm), 3.13 (d, 6H, J=5.14 ppm), 2.98 (s, 3H), 2.85 (m, 3H), 2.05 (s, 3H)p, 1.30 (m, 6H), 1.13 (d, 6H, J=6.81 Hz). Note: DCM impurity at 5.78 ppm, HDO impurity at 3.55 ppm. Chemical Formula: C₅₁H₅₇F₃N₉O₈+[M⁺] Exact Mass: 980.4277 Observed Mass [M+H]: 981.4284.

The intermediate compound (133a) was prepared as follows.

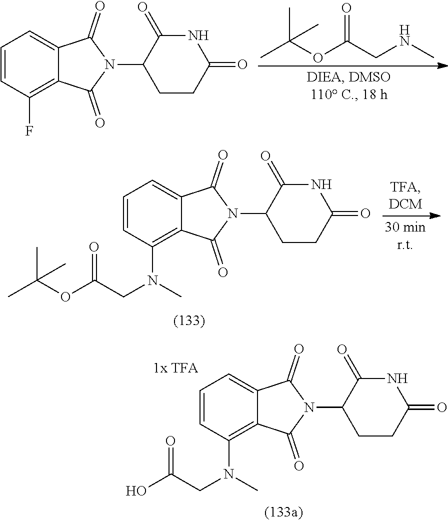

a. Synthesis of tert-butyl N-(2-(2,6-dioxopiperidin-3-yl)-1,3-dioxoisoindolin-4-yl)-N-methylglycinate (133). To a solution of 2-(2,6-dioxopiperidin-3-yl)-4-fluoroisoindoline-1,3-dione (200 mg, 0.72 mmol) in DMSO (0.5 mL) was added tert-butyl methylglycinate (110 mg, mmol) and DIEA (0.4 mL). The resulting solution was stirred in a sealed vial at 110° C. for 18 h, resulting in a brown tar. The reaction mixture was diluted with EtOAc before being washed with 10% LiCl(aq) four times. The organic layer was isolated and dried over anhydrous magnesium sulfate and purified through column chromatography with a Teledyne CombiFlash instrument (0 to 50% EtOAc in Hexanes, 4 g silica, 20 min). The product (133) was isolated as a yellow solid (207 mg, 72%). ¹H NMR (400 MHz, CDCl₃) δ 8.24 (s, 1H,), 7.57 (t, 1H, J=8.52 Hz), 7.35 (d, 1H, J=7.00 Hz), 7.16 (d, 1H, J=k8.48 Hz), 4.96 (dd, 1H, 11.93 and 5.35 Hz), 4.29 (s, 2H), 3.13 (s, 3H), 2.81 (m, 3H), 2.12 (m, 1H), 1.29 (s, 9H) Note: DCM impurity at 5.31 ppm (s, 2H), EtOAc impurity at 1.25 (t, 3H), 2.16 ppm (s, 3H), and 4.12 ppm (q, 2H).
b. Synthesis of N-(2-(2,6-dioxopiperidin-3-yl)-1,3-dioxoisoindolin-4-yl)-N-methylglycine (133a). Product (133) was dissolved in 1:1 v/v TFA/DCM and let stir for 30 minutes at ambient temperature before being blown dry under nitrogen. The resulting residue was then crashed out with cold diethyl ether and filtered to give compound (133a) (120 mg) as a vibrant yellow solid, which was used without further purification. ¹H NMR (400 MHz, DMSO) δ 11.08 (s, 1H), 7.64 (t, 1H, J=7.80), 7.27 (d, 2H, J=7.81), 5.09 (dd, 1H, J=12.81 and 5.38), 4.38 (s, 2H), 3.06 (s, 3H), 2.88 (td, 1H, J=13.27 and 9.28), 2.59 (m, 3H), 2.03 (m, 1H). ¹³C NMR (101 MHz, DMSO) δ 173.28, 171.94, 170.45, 167.46, 167.12, 149.40, 135.73, 134.15, 123.77, 113.87, 113.54, 56.93, 49.28, 41.00, 40.62, 40.41, 40.20, 39.99, 39.78, 39.58, 39.37, 31.41, 22.51. ¹⁹F NMR (376 MHz, DMSO) δ−74.82. Chemical Formula: C₁₆H₁₅N₃O₆ Exact Mass [M]: 345.0961 Observed Mass [M+H]: 346.1124.

Intermediate (131) tert-butyl (2-(2-(2-(4-(5-(2-(5-isopropyl-2-methylphenoxy)pyrimidin-4-yl)-4-(4-(trifluoromethyl)phenyl)-1H-imidazol-1-yl)piperidin-1-yl)ethoxy)ethoxy)ethyl-) carbamate was prepared as described by Divakaran, A. et al. ACS Med. Chem. Lett. (2022), 13, 1621-1627.

### Example 5

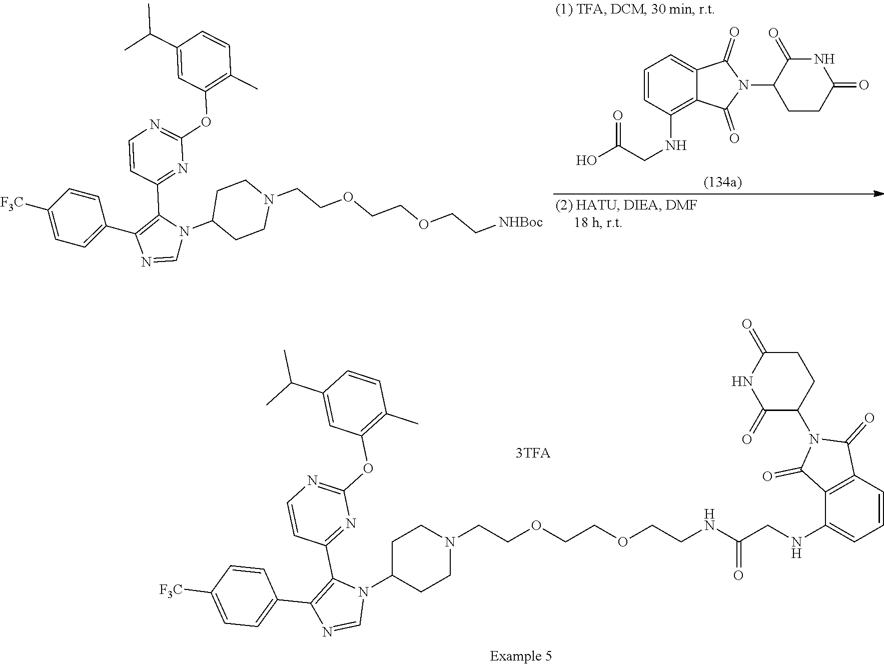 [SMILES: Cc1ccc(C(C)C)cc1Oc1nccc(-c2c(-c3ccc(C(F)(F)F)cc3)ncn2C2CCN(CCOCCOCCNC(=O)OC(C)(C)C)CC2)n1 ; O=C(O)CNc1cccc2c1C(=O)N(C1CCC(=O)NC1=O)C2=O ; Cc1ccc(C(C)C)cc1Oc1nccc(-c2c(-c3ccc(C(F)(F)F)cc3)ncn2C2CCN(CCOCCOCCNC(=O)CNc3cccc4c3C(=O)N(C3CCC(=O)NC3=O)C4=O)CC2)n1]

Intermediate (131) (30 mg, 0.040 mmol) was dissolved in a mixture of DCM (0.25 mL) and TFA (0.25 mL) and stirred for 30 minutes at ambient temperature. This mixture was then evaporated of volatiles under a stream of nitrogen before being dissolved a mixture of DMF (0.25 mL) containing excess DIEA (0.1 mL). Separately, compound (134a) (28 mg, 0.080 mmol) was dissolved in DMF (0.25 mL) and added to it was HATU (31 mg, 0.080 mmol) and DIEA (20.6 mg, 0.160 mmol). This mixture containing activated (134a) was then added dropwise to the solution containing (131), and the combined mixture was allowed to stir for 18 hours at ambient temperature. The reaction mixture was then diluted with EtOAc before being washed with 10% LiCl(aq). The organic layer was isolated and dried under magnesium sulfate before being purified through column chromatography with a Teledyne CombiFlash instrument (0 to 15% MeOH in DCM, 4 g silica, 20 min). This resulted in a vibrant yellow solid, (Example 5) (16.2 mg, 41%). ¹H NMR (400 MHz, DMSO) δ 11.11 (s, 1H), 8.64 (d, 1H, J=5.03 Hz), 8.17 (s, 2H), 7.69 (d, 2H, J=8.18 Hz), 7.60 (d, 2H, J=7.77 Hz), 7.22 (d, 1H, J=8.29 Hz), 7.12 (d, 1H, J=5.07 Hz), 7.07 (s, 3H), 6.96 (t, 1H, 5.65 Hz), 6.86 (d, 1H, 8.55 Hz), 5.08 (dd, 1H, J=12.9 and 5.42 Hz), 4.10 (br, 1H), 3.94 (d, 2H, J=5.60 Hz), 3.46 (m, 7H), 3.29 (m, 4H), 2.86 (m, 4H), 2.55 (m, obscured by DMSO peak), 2.06 (s, 4H), 1.86 (m, 4H), 1.71 (m, 1.83) 1.17 (d, 6H, J=6.86 Hz). Note: DCM impurity at 5.76 ppm. ¹³C NMR (101 MHz, DMSO) δ 173.26, 170.50, 169.17, 169.04, 167.77, 165.19, 161.56, 159.94, 151.44, 148.30, 146.28, 140.42, 136.66, 132.53, 131.62, 128.51, 127.31, 125.82, 125.55, 123.84, 120.47, 117.91, 117.81, 111.43, 110.34, 70.03, 69.42, 57.29, 54.04, 53.12, 49.04, 45.65, 40.63, 40.42, 40.21, 40.00, 39.79, 39.59, 39.38, 39.11, 33.30, 33.07, 31.45, 24.24, 22.63, 16.15. ¹⁹F NMR (376 MHz, DMSO) δ−60.92 (s, 3F). Chemical Formula: C₅₀H₅₄F₃N₉O₈ Exact Mass [M+H]: 966.4120 Observed Mass: 966.4097.

The intermediate compound (134a) was prepared as follows.

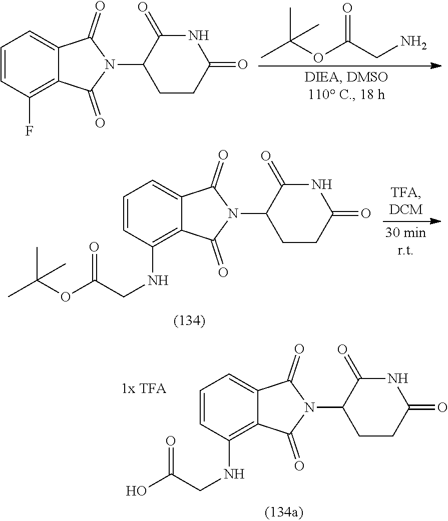

a. Synthesis of tert-butyl (2-(2,6-dioxopiperidin-3-yl)-1,3-dioxoisoindolin-4-yl)glycinate (134). To a solution of 2-(2,6-dioxopiperidin-3-yl)-4-fluoroisoindoline-1,3-dione (82 mg, 0.3 mmol) in DMSO (0.5 mL) was added tert-butyl glycinate (62 mg, 0.36 mmol) and DIEA (38 mg, 0.9 mmol). The resulting solution was stirred in a sealed vial at 110° C. for 18 hours, resulting in a brown tar. The reaction mixture was diluted with EtOAc before being washed with 10% LiCl_{(aq)} four times. The organic layer was isolated and dried over anhydrous magnesium sulfate and purified through column chromatography with a Teledyne CombiFlash instrument (0 to 50% EtOAc in Hexanesz, 4 g silica, 20 minutes). The product (134) was isolated as a yellow solid (49.9 mg, 42%). ¹H NMR (400 MHz, CDCl₃) δ 8.06 (s, 1H), 7.54 (dd, 1H, J=8.47 and 7.18 Hz), 7.19 (d, 1H, J=5.86 Hz), 6.79-6.74 (m, 2H, J=8.43 Hz), 4.96 (dd, 1H), 3.97 (s, 2H), 2.83 (m, 3H), 2.16 (m, 1H), 1.51 (s, 9H) Note: DCM impurity at 5.32 ppm, Water impurity at 1.52 ppm.
b. Synthesis of (2-(2,6-dioxopiperidin-3-yl)-1,3-dioxoisoindolin-4-yl)glycine (134a). Product (134) was dissolved in 1:1 v/v TFA/DCM and let stir for 30 minutes at ambient temperature before being blown dry under nitrogen. The resulting residue was then crashed out with cold diethyl ether and filtered to give compound 134a (72 mg) as a vibrant yellow solid, which was used without further purification. ¹H NMR (400 MHz, DMSO-d6) δ 10.86 (s, 1H), 7.34 (dd, 1H, J=8.55 and 7.07 Hz), 6.84 (d, 1H, J=6.98 Hz), 6.76 (d, 1H, J=8.56 Hz), 6.61 (t, 1H, J=5.95 Hz), 4.82 (dd, 1H, J=12.90 Hz and 5.38 Hz), 3.86 (d, 2H, J=5.24 Hz), 2.66 (m, 2H), 2.35 (m, obscured by DMSO solvent peak), 1.80 (m, 1H). ¹³C NMR (101 MHz, DMSO-d6) δ 178.04, 176.67, 175.28, 172.51, 151.05, 141.36, 122.96, 116.30, 114.86, 53.82, 48.94, 36.21, 27.36. ¹⁹F NMR (376 MHz, DMSO-d6) δ−69.43. Chemical Formula: C₁₅H₁₄N₃O₆⁺ Exact Mass [M+H]: 332.0877 Observed Mass [M+H]: 332.0861.

### Example 6

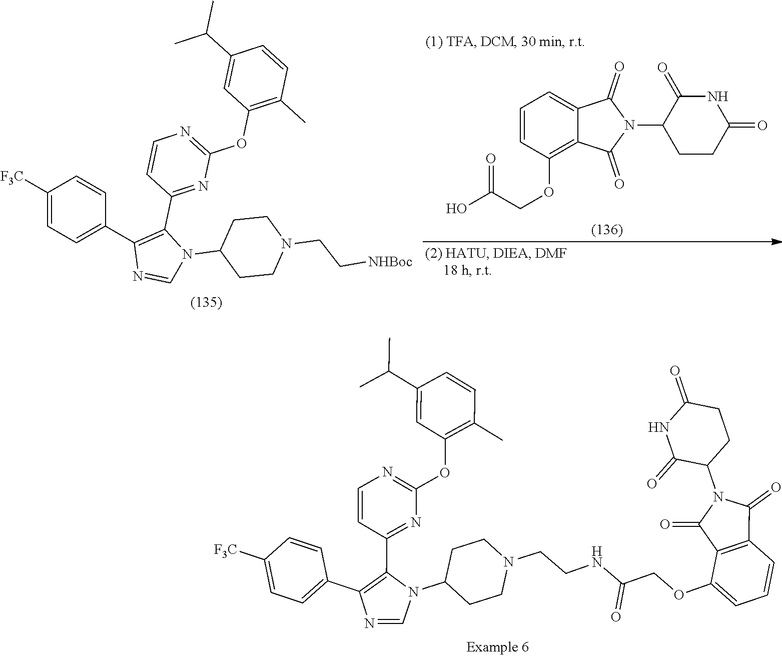 [SMILES: Cc1ccc(C(C)C)cc1Oc1nccc(-c2c(-c3ccc(C(F)(F)F)cc3)ncn2C2CCN(CCNC(=O)OC(C)(C)C)CC2)n1 ; O=C(O)COc1cccc2c1C(=O)N(C1CCC(=O)NC1=O)C2=O ; Cc1ccc(C(C)C)cc1Oc1nccc(-c2c(-c3ccc(C(F)(F)F)cc3)ncn2C2CCN(CCNC(=O)COc3cccc4c3C(=O)N(C3CCC(=O)NC3=O)C4=O)CC2)n1]

Intermediate (135) (45 mg, 0.067 mmol) was dissolved in DCM (0.25 mL) and TFA (0.25 mL) and stirred for 30 min at ambient temperature. The reaction mixture was then blown dry under a stream of nitrogen and the resulting residue was dissolved in DMF (0.25 mL). Separately, (136) was dissolved in DMF (0.25 mL) with HATU (51 mg, 0.134 mmol) and DIEA (34 mg, 0.268 mmol). The solution containing (135) was then added dropwise to the solution containing (6). The resulting reaction mixture was then let stir for 18 hours at ambient temperature before being diluted with EtOAc and washed with water. The organic layer was then washed with sat. NaCl(aq) before being isolated and dried over anhydrous magnesium sulfate. The organic layer was concentrated and purified through column chromatography with a Teledyne CombiFlash instrument (0-10% MeOH in DCM, 4 g silica, 20 min). This yielded the title compound (Example 7) as a yellow solid (34.7 mg, 60%). ¹H NMR (400 MHz, DMSO) δ 11.11 (s, 1H), 8.66 (d, 1H, J=5.05 Hz), 8.14 (s, 1H), 7.90 (dd, 1H, J=8.51 and 7.29 Hz), 7.70 (d, 2H, J=8.63 Hz), 7.62 (d, 2H, J=8.55 Hz), 7.52 (d, 1H, J=7.25 Hz), 7.45 (d, 1H, J=8.55 Hz), 7.24 (d, 1H, J=8.32 Hz), 7.15 (d, 1H, 5.12 Hz), 7.06 (m, 2H), 5.11 (dd, 1H, J=12.80 and 4.82 (s, 2H), 4.18 (br, 1H), 2.88 (m, 3H), 2.07 (m, 4H), 1.81 (br, 4H), 1.27 (m, 1.27), 1.17 (d, 6H, J=6.91 Hz). ¹⁹F NMR (376 MHz, DMSO) δ−60.92 (s, 3F). Chemical Formula: C₄₆H₄₆F₃N₈O₇+ Exact Mass [M+H]: 879.3436 Observed Mass [M+H]: 879.3482.

The intermediate compound (135) was prepared as follows.

a. Synthesis of compound (135)

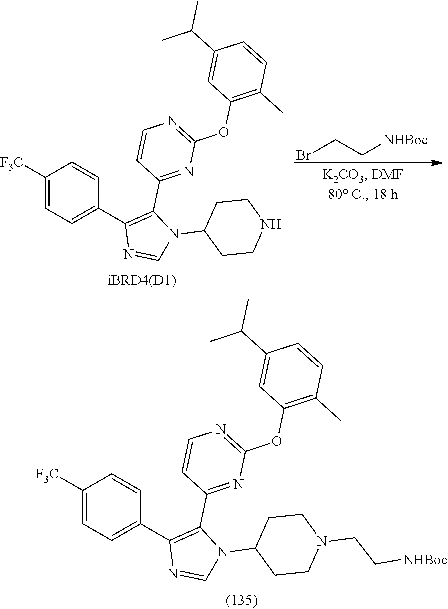 [SMILES: Cc1ccc(C(C)C)cc1Oc1nccc(-c2c(-c3ccc(C(F)(F)F)cc3)ncn2C2CCNCC2)n1 ; CC(C)(C)OC(=O)NCCBr ; Cc1ccc(C(C)C)cc1Oc1nccc(-c2c(-c3ccc(C(F)(F)F)cc3)ncn2C2CCN(CCNC(=O)OC(C)(C)C)CC2)n1]

Compound iBRD4(D1) (100 mg, 0.13 mmol) and K₂CO₃ (53.8 mg, 0.39 mmol) were stirred in DMF (3 mL) at 80° C. for 18 hours. The reaction was then allowed to cool to ambient temperature and quenched with water before being washed with EtOAc. The organic layer was collected and dried over anhydrous magnesium sulfate. The reaction mixture was then purified through column chromatography with a Teledyne CombiFlash instrument (10% MeOH in DCM, 4 g silica, 20 min), yielding (135) as a white foam (45 mg, 52%). ¹H NMR (400 MHz, CDCl₃) δ 8.43 (d, 1H, J=5.15 Hz), 7.83 (s, 1H), 7.62 (s, 4H), 7.23 (d, 1H, J=7.73 Hz), 7.09 (m, 2H), 6.87 (d, 1H, J=5.10 Hz), 5.02 (br, 1H), 4.54 (m, 1H), 3.28 (m, 2H), 2.94 (m, 3H), 2.51 (m, 2H), 2.20 (s, 3H), 1.98 (m, 6H), 1.49 (s, 9H), 1.27 (d, 6H, J=6.93 Hz). Note: DCM impurity observed at ppm. ¹³C NMR (101 MHz, CDCl₃) δ 165.22, 160.07, 155.99, 151.21, 148.37, 142.90, 137.91, 136.91, 131.29, 128.75, 127.34, 125.56, 125.53, 124.80, 123.90, 120.22, 116.47, 57.01, 53.86, 52.55, 33.56, 33.16, 28.45, 23.98, 16.19. ¹⁹F NMR (376 MHz, CDCl₃) 6-62.53 (s, 3F). Chemical Formula: C₃₆H₄₄F₃N₆O₃+ Exact Mass [M+H]: 665.3422 Observed Mass [M+H]: 665.3417.

Intermediate (136) was prepared as described by Divakaran, A., et al., (2018), https://doi.org/10.1021/acs.jmedchem.8b01248

Intermediate iBRD4(D1), was prepared as described by Divakaran, A., et al., ACS Med. Chem. Lett. (2022), 13, 10, 1621-1627. doi.org/10.1021/acsmedchemlett.2c00300.

### Example 7. Biological Evaluations

### Experimental

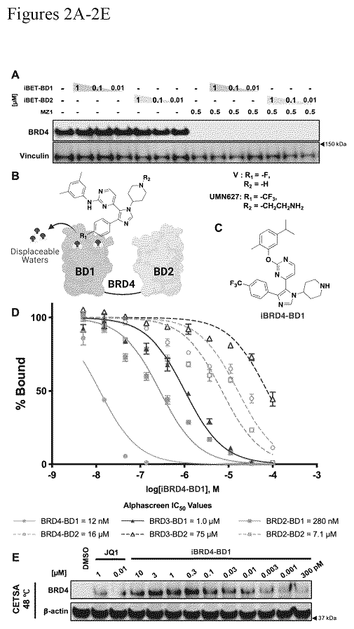

Competition experiments on pan-BET-degrader, MZ1, were performed using recently reported molecules with pan-BD1 and pan-BD2 selectivity, iBET-BD1 and iBET-BD2 (Gilan, et al., Science, 2020, 368 (6489), 387-394). In this case, neither BD1 nor BD2 inhibition was able to prevent BRD4 degradation (FIG. 2A), suggesting either BRD4 bromodomain may be a suitable target for degradation despite the preference of MZ1 to bind BRD4-BD2 while forming a ternary complex with VHL (Zengerle, M., et al., ACS Chem. Biol. 2015, 10 (8), 1770-1777). These experiments validated the strategy of domain-targeted BRD4 degradation.

In contrast to pan-BET bromodomain inhibitors, 1,4,5-trisubstituted imidazoles such as V and UMN627 preferentially bind to BD1 of BRD4 and achieve selectivity in part by displacing a network of conserved waters from the BD1 acetyl-lysine binding site. (FIG. 2B; Cui, H., et al., Angew. Chemie-Int. Ed. 2021, 60 (3), 1220-1226; and Divakaran, A., et al., J. Med. Chem. 2018, 61 (20), 9316-9334). It was hypothesized that a vacant hydrophobic area surrounding a dimethyl-aryl ring could be better occupied by bulky aliphatic groups. It was determined that replacement of the 3,5-dimethyl substituents with a 2-methyl-5-isopropyl in iBRD4-BD1 (FIG. 2C) resulted in an increase in affinity and selectivity within the BET-family (BRD4-BD1 IC₅₀=12 nM, 23-6200-fold BET selectivity; FIG. 2D). Next, the cellular binding of BRD4 in a cellular thermal shift assay (CETSA) was evaluated with previously established denaturation conditions (Divakaran, A., et al., J. Med. Chem. 2018, 61 (20), 9316-9334; and Jafari, R., et al., Nat. Protoc. 2014, 9 (9), 2100-2122). iBRD4-BD1 prevented the denaturation of BRD4 in a dose-dependent manner and showed engagement of BRD4 at concentrations above 3 nM (FIG. 2E).

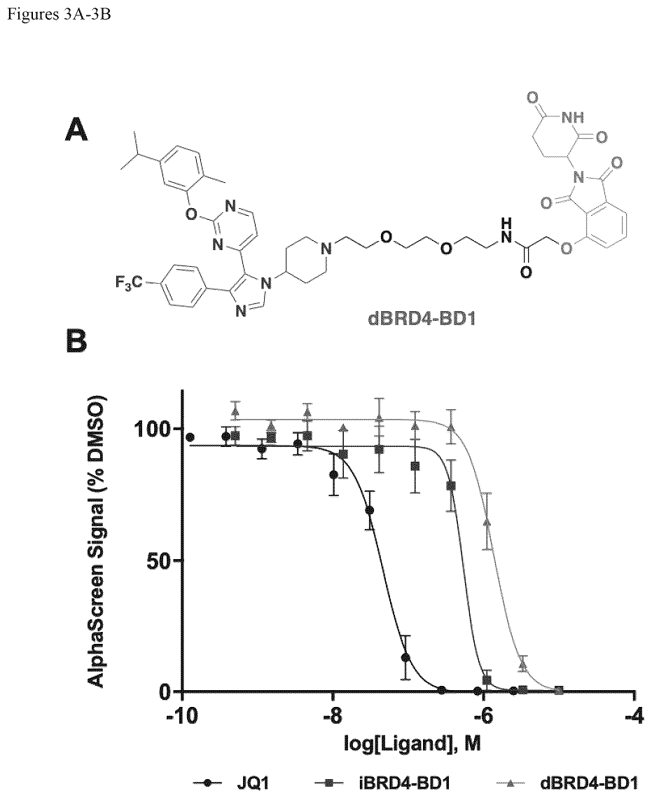

In cocrystal structures of V and UMN627 analogues, the piperidyl amine was oriented towards solvent (PDB ID: 6MH1, 6WGX). It was reasoned that this was a reasonable attachment point for E3-ligase recruiting ligands and alkylated the piperidine amine to maintain the basic nitrogen occupying an acidic region of Asp-144 and −145 around the exit vector. dBRD4-BD1 (FIG. 3A) was synthesized via conjugation of iBRD4-BD1 with a 4-hydroxythalidomide analogue via a PEG linker, which resulted in a modest loss of apparent binding to BRD4-BD1 (IC₅₀=1.36 μM, FIG. 3B). Due to poor solubility, increased DMSO was used in the assay (1%, vs. 0.1% v/v used previously), also resulting in weaker binding of iBRD4-BD1 (IC₅₀=0.47 μM vs. 0.012 μM in FIG. 2D).

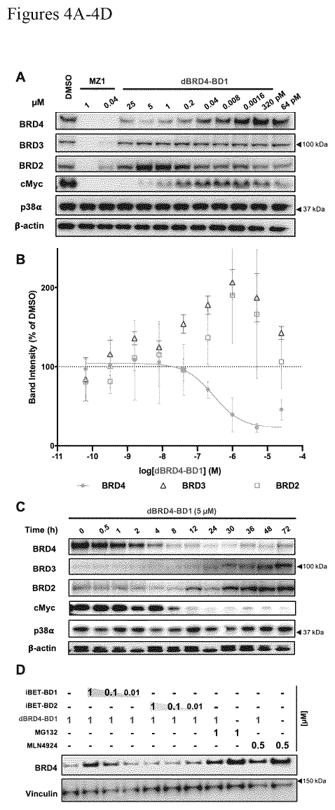

The ability to degrade BRD4 through BD1 alone was assessed. dBRD4-BD1 demonstrated selective and durable BRD4 degradation with Dₘₐₓ and DC₅₀ of 23% and 0.28 μM, respectively (FIGS. 4A and B). Upon BRD4 degradation through BRD4-BD1, degradation of BRD2 and 3 were not observed; conversely both were upregulated at concentrations where BRD4 was degraded. BRD4 degradation diminished above concentrations of 5 μM where formation of the productive ternary complex was disfavored. Overall, BRD4 was selectively degraded by dBRD4-BD1 and levels of BRD2 and 3 inversely correlated to BRD4, including at concentrations where the hook-effect was observed.

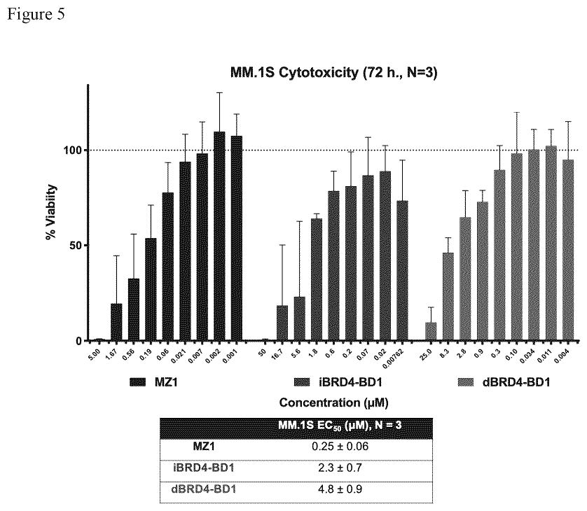

A potential benefit of the linker attachment point to iBRD4-BD1 is the effect on kinase binding. Trisubstituted imidazoles are known inhibitors of the p38a kinase and the piperidine of iBRD4-BD1 analogues (e.g., PDB ID 1OUK) occupies a region oriented towards the kinase activation loop (Gallagher, T. F., et al., Bioorganic Med. Chem. 1997, 5 (1), 49-64). Degradation of p38a was not observed at the 24-hour time-point or in a time-course study at 5 μM dBRD4-BD1 (FIG. 4C). In contrast to p38a levels in the time course study, degradation half-life of BRD4 was 3.3 hours with maximal degradation achieved after 8 hours. Concentrations of BRD2 and BRD3 increased after the 8 and 12 hour timepoints, respectively, and was followed by modest recovery in cMyc expression observed at later timepoints. Widespread effects have been observed in response to BET-inhibition and degradation (Bandopadhayay, P., et al., Nat. Commun. 2019, 10 (1); and Winter, G. E, et al., Mol. Cell 2017, 67 (1), 5-18), and the observed upregulation of proteins in response to BRD4 degradation may be broader than the set of evaluated proteins, however cellular toxicity at these later time points may be exerting a broader effect (FIG. 5).

To verify the mechanism of degradation, competition experiments were performed with domain-selective BET and proteasome inhibitors. Only iBET-BD1 was able to competitively rescue BRD4 degradation whereas iBET-BD2 had no effect (FIG. 4D). Additionally, rescue of BRD4 degradation was observed with proteasome and neddylation inhibitors, MG-132 and MLN4924. These results together indicate degradation by iBRD4-BD1 was dependent on BD1 and proceeded via ubiquitination and proteasome-dependent pathways.

### Discussion

Given the disease relevance of BRD4-specific function and to address the lack of selective BRD4 degraders, the unique properties of domain-selective BET inhibitors were levereged. This is the first instance of wt-BRD4 degradation through an individual bromodomain and may provide unique opportunities to target domain-specific BRD4 biology. Previous selective BRD4 degraders rely on optimizing pan-BET ligands for interaction kinetics (e.g., BRD4-BD2 with AT1) and linker geometries for ternary complex formation (e.g., to BRD4-BD1 with ZXH-3-26). Although in the case of BET proteins these strategies do produce selective degradation of BRD4, their major limitation is availability of the BET-ligand to bind other BET bromodomains aside from the BRD4-E3 complex which obscures biological effects and may result in toxicity. The approach presented here utilized a BRD4-BD1 specific binder to design BRD4 degraders without optimizing linker or complex interactions towards selectivity. The findings indicate BRD4 is amenable to degradation through BD1 despite detection of only a moderate ternary-complex formation by TR-FRET with dBRD4-BD1.

The observed cellular effects upon selective BRD4 degradation by dBRD4-BD1, particularly on BRD2 and 3, are unique from previously reported BRD4 degraders. Upon BRD4 degradation with AT1, ZXH-3-26 and KB02-JQ1, concentrations of BRD2 and 3 remain unchanged. The surprising effect with dBRD4-BD1 may be a potentially distinct feature of using a domain-selective BRD4 binder and arise as a cellular feedback response when other BET-bromodomains are unoccupied, such as by a pan-BET ligand. Additionally, dBRD4-BD1 has a weaker effect on cMyc expression, which is not downregulated until near maximal BRD4 degradation is achieved, likely leading to the lower observed cytotoxicity with dBRD4-BD1. Although cMyc is absent at higher dBRD4-BD1 concentrations where the hook effect is observed, inhibition of BET bromodomains alone at these concentrations, rather than BRD4 degradation, may be sufficient to affect cMyc expression.

### Methods

General procedure for AlphaScreen: Unlabeled His9-tagged BRD4-BD1 was expressed and purified as described previously (Ycas, P. D., et al., Org. Biomol. Chem. 2020, 18 (27), 5174-5182). The AlphaScreen assay procedure for BRD4-BD1 bromodomain was adapted from the manufacturers protocol (PerkinElmer, USA). Nickel chelate (Ni-NTA) acceptor beads and streptavidin donor beads were purchased from PerkinElmer (Cat. #: 6760619M). The biotinylated histone H4 KAc5,8,12,16 peptide was purchased from EpiCypher, with the sequence: Ac-SGRGK(Ac)GGK(Ac)GLGK(Ac)G-GAK(Ac)RHRKVLR-Peg(Biot). All reagents were diluted in the assay buffer (50 mM HEPES-Na+(ChemImpex), 100 mM NaCl (SigmaAldrich), 0.05% CHAPS (RPI), 0.1% BSA (SigmaAldrich), pH 7.4). The final assay concentrations (after the addition of all assay components) of 30 nM for His9-tagged BRD4-BD1 bromodomain and 50 nM for the biotinylated peptide were used. 3-fold serial dilutions were prepared with varying concentrations of the compounds and a fixed protein concentration, keeping the final DMSO concentration at 1% v/v. 5 μL of these solutions were added to a 384-well plate (ProxiPlate-384, PerkinElmer). This was followed by the addition of 5 μL of the biotinylated peptide. 5 μL of nickel chelate acceptor beads and 5 μL of streptavidin donor beads were added to each well under low light conditions (<100 lux), to a final concentration of 20 μg/mL. The plate was sealed and incubated at room temperature in the dark for 1 hour. It was then read in AlphaScreen mode using a Tecan Spark plate reader. Each compound was run in two technical replicates, with at least three biological replicates. The data was normalized against 1% DMSO (0 μM inhibitor) signal and IC₅₀ values were calculated in GraphPad Prism 5 using sigmoidal 4-parameter logistic (4PL) curve fit. BET Alphascreen was performed at Reaction Biology corp. in duplicate 10-point dose-response format, with a top-concentration of 100 μM and 3-fold dilutions.

Cell Culture: MM.1S cells were grown in a humidified 5% CO₂ environment at 37° C. Cells were cultured in RPMI 1640 media (Corning) supplemented with 10% fetal-bovine serum (FBS, Cellgro), penicillin (50 IU/mL, Cellgro) and streptomycin (50 μg/mL, Cellgro). The mixed suspension/adherent cells were subcultured at a 1:10 dilution by decanting suspended cells and dissociating adherent cells from plates in 0.25% trypsin/EDTA (Gibco) with 2 min incubation times. Cell-line authenticity was verified using the short-tandem-repeat (STR)-profiling service provided by ATCC.

CETSA: Approximately 4×10⁶ MM.1S cells were treated with the desired amounts of compound in serum supplemented RPMI-1640 media, with DMSO concentrations normalized to for all samples. Dosed cells in microcentrifuge tubes were incubated at 37° C. for 1 hour. with mild intermittent agitation. Upon completion of the incubation period, cells were pelleted at 300 X g. and rinsed in PBS, before being re-suspended in 100 μL PBS. Re-suspended cells were thermally denatured at 48° C. for 3 minutes in a heat block and subsequently equilibrated at room temperature for a further 3 minutes. Cells were pelleted at 300 X g., PBS decanted, and resuspended in PBS supplemented with 1× cOmplete Mini Protease (Roche) before being lysed over three freeze thaw cycles and centrifuged (15 minutes at 15,000 X g.). Soluble protein concentrations of supernatants were determined using the BCA protein assay kit (Pierce). Samples were normalized to the lowest total soluble-protein concentration and analyzed by western blot.

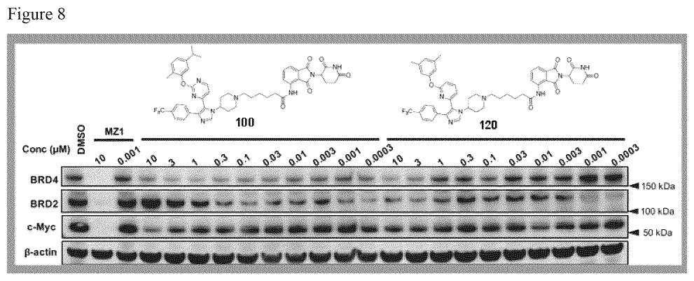

Western Blotting: MM.1S cells were seeded in 12-well plates at a density of 4×10⁶ cells per well and treated with compounds for indicated times. Cells were transferred to 2 mL microcentrifuge tubes and harvested by low-speed centrifugation at 500×g. for 5 minutes. The well was rinsed, and the cell pellet washed twice with ice-cold PBS with centrifuging. The supernatant was carefully decanted, and the cell pellet was resuspended for lysis in 100 μL of RIPA buffer (ThermoFisher Scientific) supplemented with 1× cOmplete Mini Protease (Roche) and stored on ice for 10 minutes. After high-speed centrifugation (10 min at 10,000 X g.), protein concentrations were determined by the BCA assay (ThermoFisher Scientific) and normalized by total protein content. Normalized samples were mixed with 4× NuPAGE LDS loading buffer (Invitrogen) and 10× reducing agent (Invitrogen), and heated at 95° C. for 10 minutes before separation on 3-8% Tris-Acetate gels. Proteins were transferred to PVDF membranes for 12 minutes on a BioRad Trans-Blot Turbo. Membranes were dried, blocked in TBS-T containing 5% nonfat dry milk, and subsequently incubated with TBS-T containing 5% nonfat dry milk for 4 hours at room temperature, or 16 hours at 4° C., with primary antibodies at dilutions listed below. After the membranes were washed five times with TBS-T, they were incubated with secondary antibodies for 2 hours. at room temperature. Membranes were washed five times in TBS-T and treated with SuperSignal West Dura substrates (Thermo) for 1 minute and imaged using a LiCor Odyssey Fc. Data for compounds 100 and 120 is provided in FIG. 8.

### Antibodies

| Target | Species | Manufacturer | Product No | Dilution | Conjugate |
|---|---|---|---|---|---|
| BRD4 | Rabbit | CST | E2A7X | 1:1000 | |
| BRD3 | Rabbit | Bethyl | BLR069G | 1:1000 | |
| BRD2 | Rabbit | Bethyl | BL167-2A2 | 1:1000 | |
| cMyc | Rabbit | CST | D84C12 | 1:750 | |
| p38α | Rabbit | CST | 9212S | 1:1500 | |
| β-actin | Mouse | Invitrogen | Actn05(C4) | 1:2000 | |
| Vinculin | Mouse | Thermo | 14-9777-82 | 1:2000 | |
| Tubulin | Mouse | Thermo | 236-10501 | 1:2000 | |
| Rabbit | Goat | Invitrogen | G-31460 | 1:1000 | HRP |
| Mouse | Goat | Initrogen | G-21040 | 1:2000 | HRP |
| Mouse | Goat | Invitrogen | A32729 | 1:1000 | Alexa-680 |

Viability Assays: MM. 1 S cells were seeded in 96-well plates at approximately 20 000 cells per well (0.05 mL) and dosed with increasing compound concentrations in the presence of DMSO with three technical replicates per concentration. After incubation for 69 hours at 37° C., 10 μL of the Alamar Blue reagent (Invitrogen) was added to each well and the plates were incubated for 3 hours at 37° C. Fluorescence was determined using a Synergy plate reader (BioTek, Ex.: 560 nm, Em.: 590 nm) and dose-response data were normalized to untreated and blank wells containing 0.05% DMSO in cell culture media. Data analysis was performed using GraphPad Prism.

Fluorescence-anisotropy: Experiments were carried out in 50 mM HEPES, 100 mM NaCl, and 4 mM CHAPS at pH=7.4 in 384-well plates (Corning 4511). 10 μM Fl-JQ1 stock in DMSO were diluted to 15 nM. Protein was serially diluted across the plate, after 30 minutes, anisotropy values were measured using a Tecan Infinity 500 with an excitation wavelength at 485 nm and emission at 535 nm. Anisotropy was normalized and fit using equation 1 in GraphPad Prism for direct binding experiment. In equation 1, b and c are the maximum and minimum anisotropy values; a is the concentration of Fl-JQ1(15 nM); x is the concentration of protein; and y is the observed anisotropy value in equation 1.

y=c+(b−c)*((Kd+a+x)−sqrt(Kd+a+x)∧2)−4ax)/2a equation 1

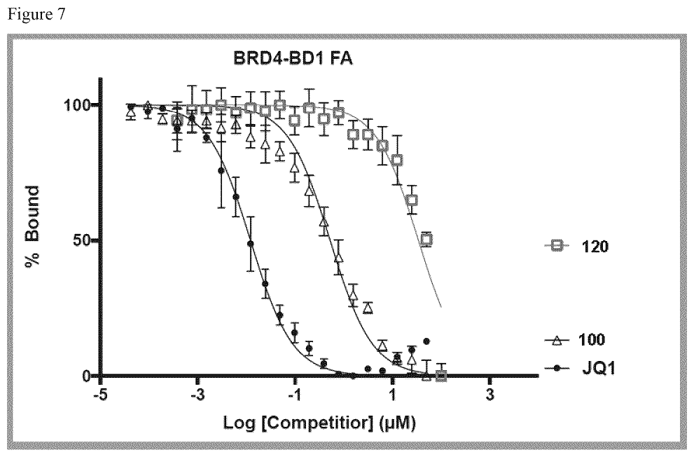

Protein concentrations of the competition experiments were determined from the direct-binding experiments at which the Fl-JQ1 is 80% bound. Using a 10 mM stock solution in DMSO, inhibitors were serially diluted from 50 μM to subnanomolar concentrations. The concentrations of protein, tracer, and other components were kept constant. Anisotropy values were fit using GraphPad Prism's [inhibitor] versus response (four parameters) function. The IC₅₀ values are reported as the mean±SEM, as determined from three independent experiments. Direct binding with Fl-JQ1 and self-competition experiments with (+)-JQ1 were carried out before competition experiments to check protein quality and assay stability. Data for compounds 100 and 120 is provided in FIG. 7.

### Example 8. Biological Evaluations

The ability of certain representative compounds degrade BRD4 was assessed (see, Table below). In particular, compound dBRD4 is shown here to degrade BRD4 with moderate potency in HEK293T cells, as well as showing a potency of 14 nM in a BRD4-dependent leukemia cell line, MV411. This supports the use of dBRD4 as a probe to selectively degrade BRD4 in multiple cell types without pan-BET inhibition. Further, modifications on the linker length support that the degradative potency of these compounds can be improved in HEK293T cells, as seen with Example 4 and Example 5. Notably, Example 5 only achieved moderate degradation levels of BRD4 compared to Example 4 and dBRD4, with Example 4 being the most potent degrader presented. Further, it was found that shorter linkers, such as Example 6, eradicate degradation potency despite achieving high levels of BRD4 degradation at higher concentrations (Dₘₐₓ=95%). Together, the presented examples support that selective inhibition of the N-terminal bromodomain of BRD4 can serve as a degrader handle to result in compounds that degrade BRD4.

Data for representative compounds is provided in the following Table.

| | HEK293 | HEK293 | MV411 | MV411 |
|---|---|---|---|---|
| Compound | (DC₅₀) | (Dₘₐₓ) | (DC₅₀) | (Dₘₐₓ) |
| dBRD4 | 500 nM | 100% | 14 nM | 85% |
| Example 4 | 105 nM | 84% | | |
| Example 5 | 290 nM | 60% | | |
| Example 6 | 810 nM | 95% | | |

All measurements at 6 hours; N = 1

### Cell Culture

HEK293T cells were grown in a humidified 5% CO₂ environment at 37° C. Cells were cultured in DMEM media (Corning) supplemented with 10% fetal-bovine serum (FBS, Cellgro), penicillin (50 IU/mL, Cellgro) and streptomycin (50 μg/mL, Cellgro). Once the adherent cells reached 85% confluency, the media was removed, and cells were treated with 0.25% trypsin/EDTA for 2 min. The trypsin was then neutralized, and the cells were pelleted by centrifugation (4000 g, 5 min) before resuspending in new media and subcultured at a 1:20 ratio.

MV4-11 cells were grown in a humidified 5% CO₂ environment at 37° C. Cells were cultured in RPMI 1640 media (Corning) supplemented with 10% fetal-bovine serum (FBS, Cellgro), penicillin (50 IU/mL, Cellgro) and streptomycin (50 μg/mL, Cellgro). Once the suspension cells reached 1.2×10⁶ cells/mL, the cells were subcultured to 500,000 cells/mL by diluting in fresh media.

### Western Blotting Protocols

HEK293T cells were seeded at 500,000 cells/well in a TC-treated 6-well plate and incubated in a 5% CO₂ environment at 37° C. for 18 hours. For MV411 cells, these were seeded at 750,000 cells/well in a TC-treated 24-well plate and incubated under similar conditions. Cells were dosed with compound for incubated times. Media was then removed and the cells were washed gently with cold PBS. To the plate was added 100 μL of RIPA buffer supplemented with 1X Roche cOmplete Mini protease inhibitor cocktail. Using a cell scraper, the cells were removed from the plate into the buffer. The cell lysate was then centrifuged at 14,000 rpm at 4° C. for 5 minutes before lysates were normalized by total soluble protein concentration using BCA protein assay kit (Peirce). Normalized samples were mixed with 4× NuPAGE LDS loading buffer (Invitrogen) and 10× reducing agent (Invitrogen), and heated at 95° C. for 10 minutes before separation on 3-8% Tris-Acetate gels. Proteins were transferred to PVDF membranes for 12 minutes on a BioRad Trans-Blot Turbo. Membranes were dried, cut, blocked in TBS-T containing 5% nonfat dry milk, and subsequently incubated with TB S-T containing 5% nonfat dry milk for 16 hours at 4° C. with an additional 2 hours at ambient temperature, with primary antibodies at dilutions listed below. After the membranes were washed five times with TBS-T, they were incubated with secondary antibodies for 2 h. at room temperature. Membranes were washed five times in TBS-T and treated with SuperSignal West Dura substrates (Thermo) for 1 minute and imaged using a LiCor Odyssey Fc. Membranes were then stained for total protein levels with SimplyBlue SafeStain (Invitrogen, Ref 465034) to confirm transfer and loading homogeneity.

The DCₘₐₓ and DC₅₀ values were determined via densitometry analysis from western blot data. Curve fitting analysis was conducted via non-linear regression using Graphpad PRISM.

| | | | | | |
|---|---|---|---|---|---|
| Target | Species | Manufacturer | Product No. | Dilution | Conjugate |

Primary Antibodies

| | | | | | |
|---|---|---|---|---|---|
| BRD4 | Rabbit | CST | E2A7X | 1:1000 | — |
| β-Actin | Mouse | Invitrogen | Actn05(C4) | 1:2000 | — |
| β-Actin | Rabbit | CST | 8457S | 1:2000 | — |

Secondary Antibodies

| | | | | | |
|---|---|---|---|---|---|
| Rabbit | Goat | Invitrogen | G-31460 | 1:2000 | HRP |
| Mouse | Goat | Invitrogen | G-21040 | 1:2000 | HRP |

Example 9. The following illustrate representative pharmaceutical dosage forms, containing a compound of formula I (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I) 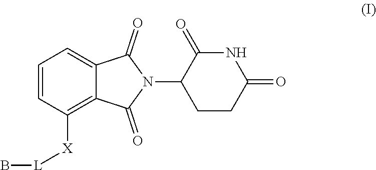
or a salt thereof wherein:
B is the residue of a molecule that selectively degrades BRD4 through its N-terminal bromodomain;
L is absent or a linker; and
X is 0 or N(R^{a}); and
R^{a} is H or (C₁-C₆)alkyl.

2. The compound of claim 1, wherein X is O or N(H).

3. The compound or salt of claim 1, wherein B is the residue of a molecule that binds to BRD4 over at least one other bromodomain by a factor of at least 100.

4. The compound or salt of claim 1, wherein L is absent.

5. The compound or salt of claim 1, wherein L comprises about 3 to about 50 atoms.

6. The compound or salt of claim 1, wherein L comprises about 3 to about 10 atoms.

7. The compound or salt of claim 1, wherein L is a branched or unbranched, saturated or unsaturated, hydrocarbon chain, having from about 1 to 100 carbon atoms, wherein one or more of the carbon atoms is optionally replaced independently by —O—, —S, —N(R^{a})—, 3-7 membered heterocycle, 5-6-membered heteroaryl or carbocycle; and wherein each chain, 3-7 membered heterocycle, 5-6-membered heteroaryl or carbocycle is optionally and independently substituted with one or more substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy; wherein each IV is independently H or (C₁-C₆)alkyl.

8. The compound or salt of claim 1, wherein L is a branched or unbranched, saturated or unsaturated, hydrocarbon chain, having from about 1 to 100 carbon atoms, wherein one or more of the carbon atoms is optionally substituted with oxo (═O); and wherein one or more of the carbon atoms is optionally replaced independently by —O—, —S, or —N(R^{a})—, wherein each R^{a} is independently H or (C₁-C₆)alkyl.

9. The compound or salt of claim 1, wherein L is a branched or unbranched, saturated or unsaturated, hydrocarbon chain, having from about 1 to 100 carbon atoms, wherein one or more of the carbon atoms is optionally substituted with oxo (═O); and wherein one or more of the carbon atoms is optionally replaced independently by —O— or —N(R^{a})—, wherein each R^{a} is independently H or (C₁-C₆)alkyl.

10. The compound or salt of claim 1, wherein L is a branched or unbranched, saturated or unsaturated, hydrocarbon chain, having from about 2 to 10 carbon atoms wherein one or more of the carbon atoms is optionally substituted with oxo (═O); and wherein one or more of the carbon atoms is optionally replaced independently by —O— or —N(R^{a})—, wherein each R^{a} is independently H or (C₁-C₆)alkyl.

11. The compound or salt of claim 1, wherein L comprises a polyethylene glycol comprising 3 to 10 repeat —CH₂CH₂O— units.

12. The compound or salt of claim 1, wherein L is selected from the group consisting of —(CH₂)ₙ—C(═O)—, and —(CH₂CH₂O)ₚ—(CH₂)ᵣ—NH—C(═O)—; wherein n is 2, 3, 4, 5, or 6; p is 2, 3, 4, or 6; and r is 2, 3, 4, 5, or 6.

13. The compound or salt of claim 1, wherein L is selected from the group consisting of —(CH₂)₅—C(═O)—, and —(CH₂CH₂O)₂—(CH₂)₂—NH—C(═O)—.

14. The compound or salt of claim 1, which is selected from: 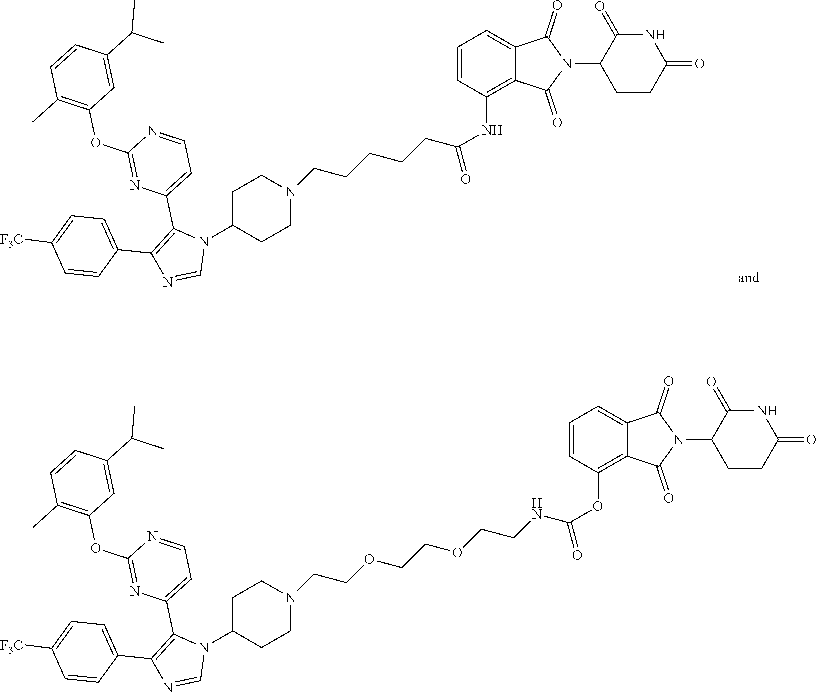 [SMILES: Cc1ccc(C(C)C)cc1Oc1nccc(-c2c(-c3ccc(C(F)(F)F)cc3)ncn2C2CCN(CCCCCC(=O)Nc3cccc4c3C(=O)N(C3CCC(=O)NC3=O)C4=O)CC2)n1 ; Cc1ccc(C(C)C)cc1Oc1nccc(-c2c(-c3ccc(C(F)(F)F)cc3)ncn2C2CCN(CCOCCOCCNC(=O)Oc3cccc4c3C(=O)N(C3CCC(=O)NC3=O)C4=O)CC2)n1]
and salts thereof.

15. The compound or salt of claim 1, which is selected from:

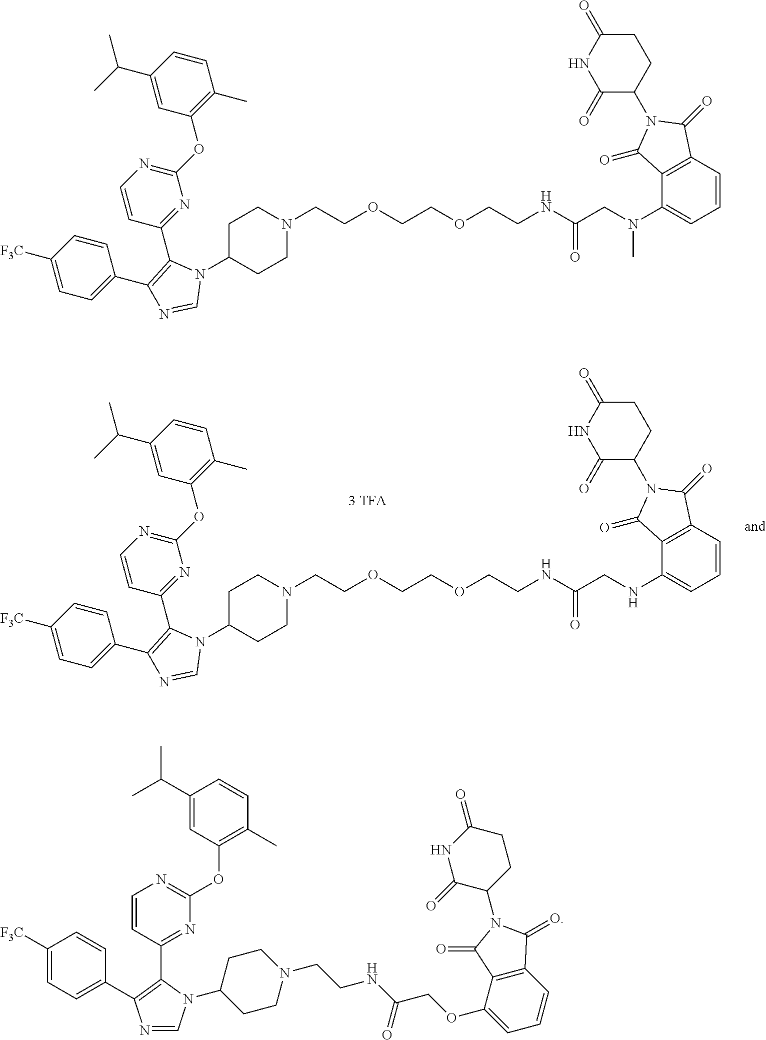 [SMILES: Cc1ccc(C(C)C)cc1Oc1nccc(-c2c(-c3ccc(C(F)(F)F)cc3)ncn2C2CCN(CCOCCOCCNC(=O)CN(C)c3cccc4c3C(=O)N(C3CCC(=O)NC3=O)C4=O)CC2)n1 ; Cc1ccc(C(C)C)cc1Oc1nccc(-c2c(-c3ccc(C(F)(F)F)cc3)ncn2C2CCN(CCOCCOCCNC(=O)CNc3cccc4c3C(=O)N(C3CCC(=O)NC3=O)C4=O)CC2)n1 ; Cc1ccc(C(C)C)cc1Oc1nccc(-c2c(-c3ccc(C(F)(F)F)cc3)ncn2C2CCN(CCNC(=O)COc3cccc4c3C(=O)N(C3CCC(=O)NC3=O)C4=O)CC2)n1]

16. A pharmaceutical composition comprising a compound of formula (I) as described in claim 1 or a pharmaceutically acceptable salt thereof and a pharmaceutically acceptable excipient.

17. A method for treating cancer in an animal, comprising administering a compound of formula (I) as described in claim 1 or a pharmaceutically acceptable salt thereof to the animal.

18. A method for treating an inflammatory condition in an animal, comprising administering a compound of formula (I) as described in claim 1 or a pharmaceutically acceptable salt thereof to the animal.

19. A method for treating alcoholic hepatitis in an animal, comprising administering a compound of formula (I) as described in claim 1 or a pharmaceutically acceptable salt thereof to the animal.

20. The compound: 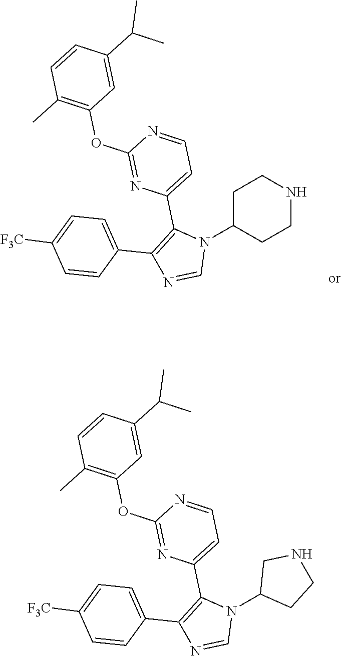 [SMILES: Cc1ccc(C(C)C)cc1Oc1nccc(-c2c(-c3ccc(C(F)(F)F)cc3)ncn2C2CCNCC2)n1 ; Cc1ccc(C(C)C)cc1Oc1nccc(-c2c(-c3ccc(C(F)(F)F)cc3)ncn2C2CCNC2)n1]
or a salt thereof.
